SEQUENCE LISTING (20 sequences)

SEQ ID NO: 1 - DNA, 4742 bp, homo sapiens
ttttctgcccttctttgctttggtggcttccttgtggttcctcagtggtgcctgcaacccctggttcacctccttccaggttctggctccttccagccatggctctcagagtccttctgttaacagccttgaccttatgtcatgggttcaacttggacactgaaaacgcaatgaccttccaagagaacgcaaggggcttcgggcagagcgtggtccagcttcagggatccagggtggtggttggagccccccaggagatagtggctgccaaccaaaggggcagcctctaccagtgcgactacagcacaggctcatgcgagcccatccgcctgcaggtccccgtggaggccgtgaacatgtccctgggcctgtccctggcagccaccaccagcccccctcagctgctggcctgtggtcccaccgtgcaccagacttgcagtgagaacacgtatgtgaaagggctctgcttcctgtttggatccaacctacggcagcagccccagaagttcccagaggccctccgagggtgtcctcaagaggatagtgacattgccttcttgattgatggctctggtagcatcatcccacatgactttcggcggatgaaggagtttgtctcaactgtgatggagcaattaaaaaagtccaaaaccttgttctctttgatgcagtactctgaagaattccggattcactttaccttcaaagagttccagaacaaccctaacccaagatcactggtgaagccaataacgcagctgcttgggcggacacacacggccacgggcatccgcaaagtggtacgagagctgtttaacatcaccaacggagcccgaaagaatgcctttaagatcctagttgtcatcacggatggagaaaagtttggcgatcccttgggatatgaggatgtcatccctgaggcagacagagagggagtcattcgctacgtcattggggtgggagatgccttccgcagtgagaaatcccgccaagagcttaataccatcgcatccaagccgcctcgtgatcacgtgttccaggtgaataactttgaggctctgaagaccattcagaaccagcttcgggagaagatctttgcgatcgagggtactcagacaggaagtagcagctcctttgagcatgagatgtctcaggaaggcttcagcgctgccatcacctctaatggccccttgctgagcactgtggggagctatgactgggctggtggagtctttctatatacatcaaaggagaaaagcaccttcatcaacatgaccagagtggattcagacatgaatgatgcttacttgggttatgctgccgccatcatcttacggaaccgggtgcaaagcctggttctgggggcacctcgatatcagcacatcggcctggtagcgatgttcaggcagaacactggcatgtgggagtccaacgctaatgtcaagggcacccagatcggcgcctacttcggggcctccctctgctccgtggacgtggacagcaacggcagcaccgacctggtcctcatcggggccccccattactacgagcagacccgagggggccaggtgtccgtgtgccccttgcccagggggagggctcggtggcagtgtgatgctgttctctacggggagcagggccaaccctggggccgctttggggcagccctaacagtgctgggggacgtaaatggggacaagctgacggacgtggccattggggccccaggagaggaggacaaccggggtgctgtttacctgtttcacggaacctcaggatctggcatcagcccctcccatagccagcggatagcaggctccaagctctctcccaggctccagtattttggtcagtcactgagtgggggccaggacctcacaatggatggactggtagacctgactgtaggagcccaggggcacgtgctgctgctcaggtcccagccagtactgagagtcaaggcaatcatggagttcaatcccagggaagtggcaaggaatgtatttgagtgtaatgatcaggtggtgaaaggcaaggaagccggagaggtcagagtctgcctccatgtccagaagagcacacgggatcggctaagagaaggacagatccagagtgttgtgacttatgacctggctctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagccttgtttccctttgagaagaattgtggcaatgacaacatctgccaggatgacctcagcatcaccttcagtttcatgagcctggactgcctcgtggtgggtgggccccgggagttcaacgtgacagtgactgtgagaaatgatggtgaggactcctacaggacacaggtcaccttcttcttcccgcttgacctgtcctaccggaaggtgtccacgctccagaaccagcgctcacagcgatcctggcgcctggcctgtgagtctgcctcctccaccgaagtgtctggggccttgaagagcaccagctgcagcataaaccaccccatcttcccggaaaactcagaggtcacctttaatatcacgtttgatgtagactctaaggcttcccttggaaacaaactgctcctcaaggccaatgtgaccagtgagaacaacatgcccagaaccaacaaaaccgaattccaactggagctgccggtgaaatatgctgtctacatggtggtcaccagccatggggtctccactaaatatctcaacttcacggcctcagagaataccagtcgggtcatgcagcatcaatatcaggtcagcaacctggggcagaggagcctccccatcagcctggtgttcttggtgcccgtccggctgaaccagactgtcatatgggaccgcccccaggtcaccttctccgagaacctctcgagtacgtgccacaccaaggagcgcttgccctctcactccgactttctggctgagcttcggaaggcccccgtggtgaactgctccatcgctgtctgccagagaatccagtgtgacatcccgttctttggcatccaggaagaattcaatgctaccctcaaaggcaacctctcgtttgactggtacatcaagacctcgcataaccacctcctgatcgtgagcacagctgagatcttgtttaacgattccgtgttcaccctgctgccgggacagggggcgtttgtgaggtcccagacggagaccaaagtggagccgttcgaggtccccaaccccctgccgctcatcgtgggcagctctgtcgggggactgctgctcctggccctcatcaccgccgcgctgtacaagctcggcttcttcaagcggcaatacaaggacatgatgagtgaagggggtcccccgggggccgaaccccagtagcggctccttcccgacagagctgcctctcggtggccagcaggactctgcccagaccacacgtagcccccaggctgctggacacgtcggacagcgaagtatccccgacaggacgggcttgggcttccatttgtgtgtgtgcaagtgtgtatgtgcgtgtgtgcaagtgtctgtgtgcaagtgtgtgcacatgtgtgcgtgtgcgtgcatgtgcacttgcacgcccatgtgtgagtgtgtgcaagtatgtgagtgtgtccaagtgtgtgtgcgtgtgtccatgtgtgtgcaagtgtgtgcatgtgtgcgagtgtgtgcatgtgtgtgctcaggggcgtgtggctcacgtgtgtgactcagatgtctctggcgtgtgggtaggtgacggcagcgtagcctctccggcagaagggaactgcctgggctcccttgtgcgtgggtgaagccgctgctgggttttcctccgggagaggggacggtcaatcctgtgggtgaagacagagggaaacacagcagcttctctccactgaaagaagtgggacttcccgtcgcctgcgagcctgcggcctgctggagcctgcgcagcttggatggagactccatgagaagccgtgggtggaaccaggaacctcctccacaccagcgctgatgcccaataaagatgcccactgaggaatgatgaagcttcctttctggattcatttattatttcaatgtgactttaattttttggatggataagcttgtctatggtacaaaaatcacaaggcattcaagtgtacagtgaaaagtctccctttccagatattcaagtcacctccttaaaggtagtcaagattgtgttttgaggtttccttcagacagattccaggcgatgtgcaagtgtatgcacgtgtgcacacacaccacacatacacacacacaagcttttttacacaaatggtagcatactttatattggtctgtatcttgctttttttcaccaatatttctcagacatcggttcatattaagacataaattactttttcattcttttataccgctgcatagtattccattgtgtgagtgtaccataatgtatttaaccagtcttcttttgatatactattttcattctcttgttattgcatcaatgctgagttaataaatcaaatatatgtcatttttgcatatatgtaaggataa

SEQ ID NO: 2 - protein, 1152 aa, homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 3 - DNA, 5101 bp, Homo sapiens
cactcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacccccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtgagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcccccactccagataaagcttcagttatatctca

SEQ ID NO: 4 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 5 - DNA, 42 bp, Homo sapiens
ttcaggcagaacactggcatgtgggagtccaacgctaatgtc

SEQ ID NO: 6 - protein, 14 aa, Homo sapiens
FRQNTGMWESNANV

SEQ ID NO: 7 - DNA, 42 bp, Homo sapiens
ttcaggcagaacactggcacgtgggagtccaacgctaatgtc

SEQ ID NO: 8 - protein, 14 aa, Homo sapiens
FRQNTGTWESNANV

SEQ ID NO: 9 - DNA, 42 bp, Homo sapiens
ttcaagtcagaaaaggggcgcaacaagttcgtgaccgtgcag

SEQ ID NO: 10 - protein, 14 aa, Homo sapiens
FKSEKGRNKFVTVQ

SEQ ID NO: 11 - DNA, 42 bp, Homo sapiens
ttcaagtcagaaaaggggggcaacaagttcgtgaccgtgcag

SEQ ID NO: 12 - protein, 14 aa, Homo sapiens
FKSEKGGNKFVTVQ

SEQ ID NO: 13 - DNA, 20 bp, Artificial
  Primer
ccaaaacggccacctcggaa

SEQ ID NO: 14 - DNA, 20 bp, Artificial
  Primer
ccgtccggcccacgggtagc

SEQ ID NO: 15 - DNA, 23 bp, Artificial
  Primer
gaatgcacttcacctctcagacc

SEQ ID NO: 16 - DNA, 22 bp, Artificial
  Primer
gggcgcctctgtttgcacattc

SEQ ID NO: 17 - DNA, 21 bp, Artificial
  Primer
gctctcactgccctcctctgc

SEQ ID NO: 18 - DNA, 19 bp, Artificial
  Primer
ggatacttcgctgtccgac

SEQ ID NO: 19 - DNA, 24 bp, Artificial
  Primer
gccttaaaggagaccagggggaac

SEQ ID NO: 20 - DNA, 23 bp, Artificial
  Primer
cccttgaggaggagacgatggac

(19) United States
(12) Patent Application Publication
(10) Pub. No.: US 2012/0027750 A1
(43) Pub. Date: Feb. 2, 2012

(54) METHOD AND COMPOSITIONS FOR CANCER PROGNOSIS

(75) Inventors: Hervé Watier, Ballan Mire (FR); Guillaume Cartron, Combaillaux (FR)

(73) Assignees: Centre Hospitalier Regional Universitaire de Tours, Tours Cedex 9 (FR); Universite Francois Rabelais de Tours, Tours Cedex 1 (FR)

(21) Appl. No.: 13/145,543

(22) PCT Filed: Dec. 4, 2009
(86) PCT No.: PCT/EP2009/066463

§ 371 Date: Oct. 4, 2011

(30) Foreign Application Priority Data
Dec. 8, 2008 (GB) 08223455

(51) Int. Cl. A61K 39/395; A61P 35/00; C12Q 1/68

(52) U.S. Cl. 424/133.1; 435/6.11; 424/173.1

(57) ABSTRACT

Methods are provided to determine the cancer prognosis of subjects and/or to adapt the treatment protocol of subjects having or susceptible to cancer. Embodiments include the steps of determining in vitro the genotype of said subject at a polymorphism in the C3-ITGAM axis, making a cancer prognosis of the subject based on said genotype and selecting an anti-cancer treatment for the subject.

The present invention relates to methods and compositions to evaluate or assess cancer prognosis for a subject. More particularly, the invention provides methods to determine the cancer prognosis of subjects, or to adapt the treatment protocol of subjects having or susceptible to cancer. The invention can be used in particular for patients treated with therapeutic antibodies that target and deplete cancer cells.

### INTRODUCTION

Cancer remains to be one of the most deadly threats to human health. In the U.S., cancer affects nearly 1.3 million new patients each year, accounting for approximately 1 in 4 deaths. It is also predicted that cancer may surpass cardiovascular diseases as the number one cause of death in the coming years. Although there have been significant advances in the medical treatment of certain cancers, the overall 5-year survival rate for all cancers has improved only by about 10% in the past 20 years.

Cancer treatment, such as chemotherapy, radiation and/or surgery, has associated risks, and it would be useful to be able to optimally select patients most likely to benefit. Prognostic testing is useful to, for example, identify patients with poor prognoses such that a more aggressive, higher risk treatment approach is identified, and to identify patients with good prognoses for whom risky therapy would not provide enough benefit to warrant the risks. Thus, despite the existence of treatments have efficacy, many patients relapse, and moreover experience different outcomes following a treatment, including duration of survival or disease stabilization, degree of tumor regression for example. Such responses are often expressed as duration of progression free survival (PFS) or duration of overall survival (OS), or whether an objective response (OR) or complete response (CR) is obtained. There is an urgent need for new cancer prognostic factors that could identify patients likely to have poor prognostics, including when treated with e.g. lower risk treatments, so that these patients could be treated using more potent regimens.

For example, the treatment of B lymphoproliferative malignancies, particularly non-Hodgkin's lymphomas (NHL) including mostly follicular lymphoma (FL) and diffuse large B-cell lymphoma (DLBCL), has been modified by use of rituximab (Mabthera®, Rituxan®), which is a chimeric anti-CD20 IgG1 monoclonal antibody made with human γ1 and κ constant regions linked to murine variable domains. However, patients typically relapse following treatment with rituximab as single agent. Patients are therefore often treated with chemotherapy in addition to rituximab as their first-line of therapy. However, chemotherapy has adverse side-effects. In the case of B lymphoproliferative malignancies, prognostic markers would be useful to identify patients with poor prognostics such that these patients could be treated with a more potent treatment, e.g. chemotherapy and rituximab. Patents with good prognostics could be treated with rituximab regimens adapted to their genotype. As reviewed in Cartron G, Watier H, Golay J, Solal-Celigny P. From the bench to the bedside: ways to improve rituximab efficacy. Blood. 2004; 104:2635-2642, even with the effective cancer therapies such as rituximab, there remains a need for means to improve cancer prognosis and treatment.

### SUMMARY OF THE INVENTION

The invention is based on the finding of a correlation between polymorphisms affecting the C3-ITGAM axis and a subject's cancer prognosis. ITGAM, also referred to as CD11b, is the α chain of the complement receptor 3 (CR3, α_{M}β₂, Mac-1, CD11b/CD18), an integrin expressed on effector cells such as granulocytes, macrophages or NK cells, and CD11b is encoded by ITGAM (for “Integrin alpha M”) gene. More specifically, the invention shows that the genotype of CD11b (the α chain of CR3) and its ligand C3 is predictive or indicative with the subject's cancer prognosis, including when a subject having cancer has been treated with an anti-cancer therapy, e.g. anti-CD20 antibody.

In one aspect the invention involves detecting, in a subject or biological sample, a polymorphism or a locus closely linked thereto, the polymorphism being in an ITGAM or C3 gene, wherein the polymorphism is associated with cancer prognosis. The methods may further include correlating an allele of the ITGAM or C3 polymorphism to cancer prognosis, optionally correlating said allele to response to a therapy, e.g. a therapeutic antibody. Preferably, the ITGAM polymorphism is in the domain of ITGAM that influences interaction with C3b. Preferably, the C3 polymorphism is in the domain of C3 that influences the binding of C3 to a cell membrane. Preferably, the ITGAM polymorphism is in the domain of ITGAM containing residues Asp³⁹⁸ to Thr⁴⁵¹. Preferably the polymorphism is in amino acid position 425 for ITGAM and in position 80 for C3.

Accordingly, in one aspect the invention provides methods for evaluation of a subject having or suspected of having cancer, the method comprising (a) determining the subject's ITGAM or C3 genotype, and (b) making a cancer prognosis of the subject based on the ITGAM or C3 genotype. Determining the subject's ITGAM or C3 genotype can be carried out for example by obtaining a biological sample from the subject and detecting the presence of a nucleotide or amino acid at a particular polymorphic site in a ITGAM or C3 gene or protein, respectively. Determining the subject's ITGAM or C3 genotype can also be carried out by accessing a database containing the subject's genotype information. The method may also comprise comparing a subject's ITGAM or C3 genotype with control or reference genotype(s), and making a cancer prognosis of the subject based on the comparison in, wherein the subject's ITGAM or C3 genotype relative to the control or reference is prognostic for cancer progression in the subject.

In some embodiments, a cancer prognosis, a prognostic for cancer or cancer progression comprises providing the forecast or prediction of (prognostic for) any one or more of the following: duration of survival of a subject susceptible to or diagnosed with a cancer, duration of recurrence-free survival, duration of progression free survival of a subject susceptible to or diagnosed with a cancer, response rate to treatment in a subject or group of subjects susceptible to or diagnosed with a cancer, and/or duration of response, degree of response, or survival following treatment in a subject or a group of subjects susceptible to or diagnosed with a cancer. Preferably the treatment comprises administering a therapeutic antibody. In some embodiments, the presence of a favorable allele indicates that the duration of survival is forecast or predicted to be increased. In some embodiments, the presence of an unfavorable allele indicates that the duration of survival is forecast or predicted to be decreased. In some embodiments, the presence of a favorable allele indicates that the duration of recurrence-free survival is forecast or predicted to be increased. In some embodiment, the presence of an unfavorable allele indicates that the duration of recurrence-free survival is forecast or predicted to be decreased. In some embodiments, the presence of a favorable allele indicates that the response rate is forecast or predicted to be increased. In some embodiments, the presence of an unfavorable allele indicates that the response rate is forecast or predicted to be decreased. In some embodiments, the presence of a favorable allele indicates that the duration of response is predicted or forecast to be increased. In some embodiments, duration of response is predicted or forecast to be decreased.

In another aspect, the invention also provides methods to select or identify patients having favourable or unfavorable cancer prognostics, and optionally further treating these patients according to their cancer prognostics. In one aspect the invention provides methods for selection of treatment for a subject having or suspected of having cancer, the methods comprising (a) determining the subject's ITGAM or C3 genotype, (b) making a cancer prognosis of the subject based on the ITGAM or C3 genotype; and (c) subsequent to steps (a)-(b), selecting an anti-cancer treatment for the subject, wherein the selection of treatment is based on the prognosis determined in step (b). Optionally, the method further comprises step (d), treating the subject with the anti-cancer treatment selected in step (c).

In some aspects of any of the embodiments herein, the cancer prognostic is response rate to treatment in a subject or group of subjects susceptible to or diagnosed with cancer, optionally duration of response, degree of response, or survival following treatment. Optionally, said treatment (e.g. anti-cancer treatment) comprises administration of a therapeutic antibody, optionally wherein the antibody comprises an Fc portion, optionally wherein the antibody is of the G1 or G3 subtypes, optionally wherein the antibody is specific for CD20 (an anti-CD20 antibody), optionally wherein the antibody is rituximab, or the antibody is directed against (specific for) an antigen selected from the group consisting of CD3, CD4, CD5, CD6, CD8, CD14, CD15, CD16, CD19, CD20, CD21, CD22, CD23, CD25, CD32B, CD30, CD33, CD37, CD38, CD40, CD40L (CD154), CD44 and its splice variant CD44v6, CD46, CD52, CD54, CD56, CD59, CD70, CD74, CD79, CD80, CD122, CD126, CD133, CD138, CD137, CD152 (CTLA-4), CD200, CD317 (HM1.24), human leukocyte antigen (HLA)-DR, Flt3, CCR4, BR3/Blys3R, EpCAM, MUC1, MCAM/MUC18, podoplanin, CEA (carcinoembryonic antigen), PDGFR, GD2, GD3, GM2 and GM3 gangliosides, LeY, PSMA (prostate specific membrane antigen), PSCA (prostate stem cell antigen), A33, CAIX/MN, TRAIL-R1 and TRAIL-R2, HMW-MMA (human high molecular weight melanoma associated antigen), BCMA (B-cell maturation antigen), FRA (folate receptor α)/gp38, tenascin, phosphatidylserine, GFAP (glial fibrillary acidic protein), AMVB1, Tn-antigen, ICAM1, IL6-R, HGFR, EGFR, IGF-1R, a member of the human EGF-like receptor family such as HER-2/neu, HER-3, HER-4 or a heterodimeric receptor comprised of at least one HER subunit, CRIPTO antigens (e.g. CRIPTO-1, CRIPTO-3), a member of FGF receptor family including FGFR1 and FGFR3. Optionally, the cancer is a B-cell lymphocytic leukemia, or optionally any other subtype of B-cell disorder, a non-Hodgkins lymphoma (NHL), a multiple myeloma, a lung cancer, breast cancer, or a colon cancer.

Accordingly, in another aspect, the invention provides a method of assessing the response of a subject to a therapeutic antibody treatment, or for selecting a subject for therapeutic antibody treatment, the method comprising determining in vitro the polymorphism in position 425 for ITGAM or the polymorphism in position 80 for C3 of said subject.

In some embodiments, a favourable ITGAM or C3 genotype indicates that a subject is suited for treatment with a reduced intensity treatment compared to a subject with an unfavourable cancer prognostic. The reduced intensity treatment may comprise for example treatment with the standard therapeutic approach that does not distinguish between ITGAM and C3 genotypes, treatment with a sole therapeutic agent or therapeutic approach, treatment with a therapeutic antibody, treatment with a therapeutic antibody in the absence of one or more selected adjuvants (e.g. an adjuvant having toxicity), or treatment with a therapeutic antibody as sole anti-cancer agent. In some embodiments, an unfavourable ITGAM or C3 genotype indicates that a subject is suited for treatment with an increased intensity treatment compared to a subject with a favourable cancer prognostic, e.g. treatment with a multiple therapeutic agents or therapeutic approaches, treatment with an chemotherapy, for example chemotherapy in addition to or instead of a therapeutic antibody, treatment with a therapeutic antibody and an adjuvant. The methods of the invention optionally further comprise administering to the subject the selected cancer treatment, e.g. a reduced or increased intensity treatment.

The invention also relates to compositions and kits suitable to perform the invention. The invention may as well be used in clinical trials or experimental settings, to assess or monitor a subject's response to a treatment. The invention also relates to use of any of pharmaceutical compositions comprising the therapeutic agents described herein (e.g. therapeutic antibodies, therapeutic antibodies having increased potency, optionally with or without an adjuvant, chemotherapy) for use in treating subjects with favourable or unfavourable prognostics based on their ITGAM or C3 genotype, optionally as determined or assessed using any of the embodiments described herein.

In preferred embodiments, determining in vitro the ITGAM genotype and/or the presence of a polymorphism in an ITGAM polypeptide comprises determining in vitro the presence of a polymorphism at amino acid position 425 of ITGAM. More specifically, determining in vitro the ITGAM genotype of a subject at amino acid position 425 of ITGAM comprises determining the amino acid residue at position 425 of ITGAM (or corresponding codon in the ITGAM gene), a methionine (M) at position 425 being indicative of a favorable cancer prognostic and a threonine (T) at position 425 being indicative of an unfavorable cancer prognostic. Preferably, heterozygosity or homozygosity for a threonine (T) at position 425 is indicative of an unfavorable cancer prognostic, and homozygosity for a methionine is indicative of a favorable cancer prognostic

In preferred embodiments, determining in vitro the C3 genotype and/or the presence of a polymorphism in a C3 polypeptide comprises determining in vitro the presence of a polymorphism at amino acid position 80 of C3. More specifically, determining in vitro the C3 genotype of a subject at amino acid position 80 of C3 comprises determining the amino acid residue at position 80 of C3 (or corresponding codon in the C3 gene), an arginine (R) at position 80 being indicative of a favourable cancer prognostic and a glycine (G) at position 80 being indicative of an unfavourable cancer prognostic. Preferably, homozygosity for a glycine at position 80 is indicative of an unfavourable cancer prognostic, and heterozygosity or homozygosity for an arginine is indicative of a favourable cancer prognostic.

Another object of this invention is a method of treating a subject having or suspected of having cancer, comprising: determining a subject's ITGAM or C3 genotype, and administering to the subject a treatment regimen based upon the subject's genotype, wherein i) if the subject is homozygous for the M allele at the amino acid at position 425 of the mature ITGAM protein, or has an R allele at the amino acid at position 80 of the mature C3 protein, then selecting or administering a first treatment regimen, and ii) if the subject has a T allele at the amino acid at position 425 of the mature ITGAM protein, or homozygous for G allele at the amino acid at position 80 of the mature C3 protein, then selecting or administering a second treatment regimen which is different from the first treatment regimen, to thereby treat the cancer. Optionally the first treatment regimen is a standard treatment regimen or a reduced intensity treatment regimen. Optionally the second treatment regimen is a standard treatment regimen or an increased intensity treatment regimen. In certain examples, when the first treatment regimen is a reduced intensity treatment regimen, the second treatment regimen is a standard or increased intensity treatment regimen; when the second treatment regimen is an increased intensity treatment regimen, the first treatment regimen may be a standard or reduced intensity treatment regimen.

In another aspect, the method of treating a subject includes selecting a subject based upon the subject being homozygous for the M allele at the amino acid at position 425 of the mature ITGAM protein, or has an R allele at the amino acid at position 80 of the mature C3 protein, or a nucleotide, allele or combination of alleles at loci in linkage disequilibrium with the amino acid at position 425 of the mature ITGAM protein or the amino acid at position 80 of the mature C3 protein, and administering to the subject an anti-cancer treatment. Optionally the treatment is a standard or decreased intensity treatment.

Also encompassed is the use of any of the preceding treatment regimens, for the treatment of a subject homozygous for the M allele at the amino acid at position 425 of the mature ITGAM protein, or having an R allele at the amino acid at position 80 of the mature C3 protein, or a nucleotide, allele or combination of alleles at loci in linkage disequilibrium with the amino acid at position 425 of the mature ITGAM protein or the amino acid at position 80 of the mature C3 protein.

In another aspect, the method of treating a subject includes selecting a subject based upon the subject having a T allele at the amino acid at position 425 of the mature ITGAM protein, or being homozygous for G allele at the amino acid at position 80 of the mature C3 protein, or a nucleotide, allele or combination of alleles at loci in linkage disequilibrium with the amino acid at position 425 of the mature ITGAM protein or the amino acid at position 80 of the mature C3 protein, and administering to the subject an anti-cancer treatment. Optionally the treatment is a standard or increased intensity treatment.

Also encompassed is the use of any of the preceding treatment regimens, for the treatment of a subject having a T allele at the amino acid at position 425 of the mature ITGAM protein, or being homozygous for G allele at the amino acid at position 80 of the mature C3 protein, or a nucleotide, allele or combination of alleles at loci in linkage disequilibrium with the amino acid at position 425 of the mature ITGAM protein or the amino acid at position 80 of the mature C3 protein.

In another embodiment, the invention provides a method for optimizing clinical trial design for a treatment regimen, wherein the method comprises determining in vitro the ITGAM or C3 genotype and/or the presence of a polymorphism in an ITGAM or C3 polypeptide of said subject; and allowing classification of the subjects in at least two subsets, wherein a first subset may be treated with a first anti-cancer treatment and a second subject is treated with a second anti-cancer treatment, wherein the first and second anti-cancer treatment differ, e.g. in the nature of the treatment, the composition administered, or the dose and/or administration schedule used for a composition.

The polymorphisms can be detected by any available method, including amplification, hybridization to a probe or array, or the like. In one specific embodiment, detection includes amplifying the polymorphism, linked locus or a sequence associated therewith (e.g., flanking sequences, transcribed sequences or the like) and detecting the resulting amplicon. For example, in one embodiment, amplifying includes a) admixing an amplification primer or amplification primer pair with a nucleic acid template isolated from the organism or biological sample. The primer or primer pair can be complementary or partially complementary to a region proximal to or including the polymorphism or linked locus, and are capable of initiating nucleic acid polymerization by a polymerase on the nucleic acid template. The primer or primer pair is extended in a DNA polymerization reaction comprising a polymerase and the template nucleic acid to generate the amplicon. In certain aspects, the amplicon is optionally detected by a process that includes hybridizing the amplicon to an array, digesting the amplicon with a restriction enzyme, or real-time PCR analysis. Optionally, the amplicon can be fully or partially sequenced, e.g., by hybridization. Typically, amplification can include performing a polymerase chain reaction (PCR), reverse transcriptase PCR (RT-PCR), or ligase chain reaction (LCR) using nucleic acid isolated from the organism or biological sample as a template in the PCR, RT-PCR, or LCR. Other technologies can be substituted for amplification, e.g., use of branched DNA probes.

### DETAILED DESCRIPTION OF THE FIGURES

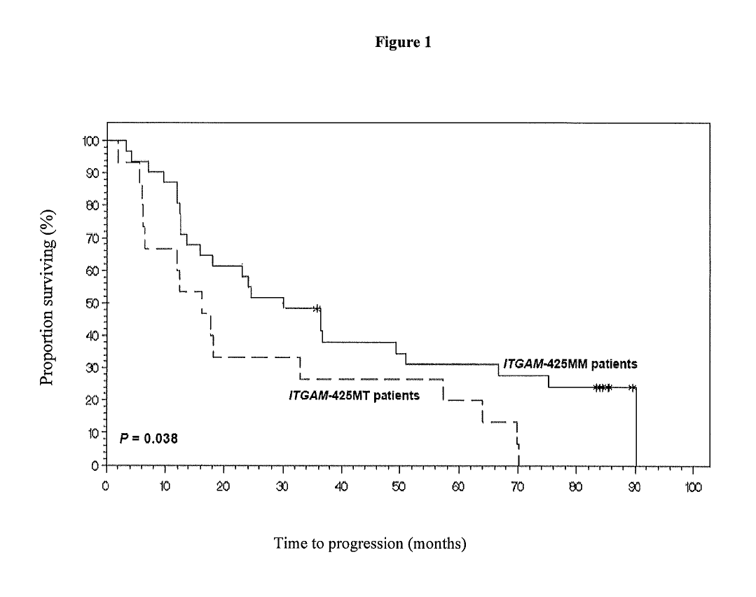

FIG. 1 shows median time of PFS (median follow-up: 84 months) was 30 months (CI 95%: 16-51 months) for homozygous ITGAM-425M and 16 months (CI 95%: 6-23 months) for heterozygous patients (P=0.038) whereas PFS was not influenced by C3-80RG, ITGAM-1130PS and C1QA-70_{GA} polymorphisms (data not shown). The Cox regression analysis confirmed the previously described influence of BCL2-JH rearrangement disappearance in bone marrow at day 50 (Colombat P, et al. Blood. 2001; 97:101-106) (P=0.01; HR 0.1, CI 95%: 0.02-0.65] and showed that ITGAM-425MT polymorphism predicted significantly the PFS (P=0.001; HR: 9.1, CI 95%: 2.4-33.9).

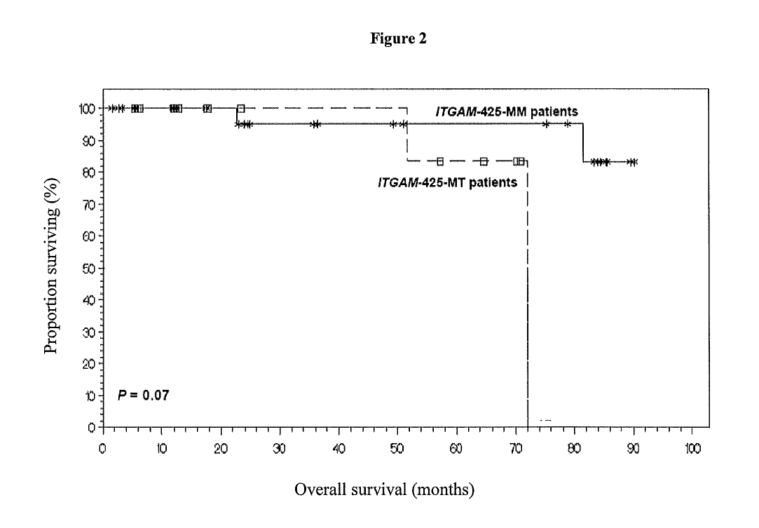

FIG. 2 shows median time of OS was 72 months (CI 95%: 52-72) for heterozygous ITGAM-425MT patients and was not reached for homozygous ITGAM-425M patients (P=0.07).

### DETAILED DESCRIPTION OF THE INVENTION

The inventor's group has previously demonstrated that follicular lymphoma (FL) patients homozygous for the FCGR3A-158V allele (encoding the FcγRIIIa allotype of highest affinity for IgG1) have a better response to rituximab (Cartron G, et al. Blood. 2002; 98:754-758. Because of FcγRIIIa is expressed by monocytes and NK cells, these results strongly suggested an involvement of antibody-dependant cell-mediated cytotoxicity (ADCC) in rituximab activity in human. Complement-dependant cytotoxicity (CDC) is also induced by rituximab on B lymphoma cell lines (Flieger D, et al. Cell Immunol, 2000; 204:55-63; Harjunpaa A, et al. Scand J. Immunol. 2000; 51:634-641; Reff M E, et al. Blood 1994; 83:435-445) and fresh B lymphoma cells. (Golay et al. Blood 2001; 98:3383-3389; Golay J, et al. Blood. 2000; 95:3900-3908; Bellosillo B et al. Blood 2001; 98:2771-2777; Weng W K and Levy R. Blood 2001; 98:1352-1357). The demonstration that rituximab is unable to cure C1q-deficient mice inoculated with syngenic lymphoma cells (EL4) transduced with human CD20 provides the first in vivo argument showing that complement activation is required (Di Gaetano N, et al. J. Immunol. 2003; 171:4251-4257) It has been thought that interaction between C3b and CR3 enhance FcγR-mediated effector-cell binding and cytotoxicity. (Zhou M J, et al. J Cell Biol. 1994; 125:1407-1416; Perlmann H, J Exp Med. 1981; 153:1592-1603; Ehlenberger A G and Nussenzweig V. J Exp Med. 1977; 145:357-371).

The complement system consists of classical, lectin and alternative pathways which converge and ultimately generate a large amount of C3b, the main effector molecule of the complement system. C3b molecules are generated by the cleavage of C3 protein generating C3a anaphylatoxin and the major fragment C3b. C3b binds to the C3 convertase to form C5 convertase, leading to the generation of the membrane attack complex which kills target cells by disrupting of the cell membrane. C3b also acts as opsonin and interact with different complement receptors (CRs) expressed by immune cells, including CR3. ITGAM (for “Integrin alpha M”), also referred to as CD11b, is the α chain of the integrin CR3 (α_{M}β₂, Mac-1, CD11b/CD18) expressed on effector cells such as granulocytes, macrophages or NK cells.

Two allotypic forms of C3 have been described on the basis of electrophoretic motility. (Alper C A et al. J Clin Invest. 1968; 47:2181-2191) At molecular level, there is a single-nucleotide polymorphism (SNP; C to G) at nucleotide 364 leading to either an arginine (R) or a glycine (G) at amino-acid position 80 (Botto M, et al. J Exp Med. 1990; 172:1011-1017). Functional consequences of such polymorphism on its ability to bind CRs remains controversial (Arvilommi H. Nature. 1974; 251:740-741; Bartok I, and Walport M J. J. Immunol. 1995; 154:5367-5375) but an association between C3-80-RG polymorphism (called also C3-S/F to refer to the slow or fast electrophoretic motility) and IgA nephropathy, (Rambausek M C, et al. Nephrol Dial Translant. 1987; 2:208-211) systemic vasculitis, (Finn J E, et al. Nephrol Dial Translant. 1994; 9:1564-1567) mesangiocapillary glomerulonephritis (Finn J E, et al. Clin Exp Immunol. 1993; 91:410-414; McLean R H and Winkelstein J A. J. Pediatr. 1984; 105:179-188) and more recently late-renal transplantation outcome (Brown K M, et al. N Engl J. Med. 2006; 354:2014-2023) suggest that the two alleles might have functional differences. Interaction between ITGAM and C3b involves two separate domains located in the α_{M}I-domain and the α_{M}β-propeller domain repeats of the ITGAM (Yalamanchili P, et al. J Biol Chem. 2000; 275:21877-21882; Diamond M S, et al. J Cell Biol. 1993; 120:1031-1043). Recent study has however pointed out the critical role of residues Asp³⁹⁸ to Thr⁴⁵¹ located within the α_{M}β-propeller in this interaction (Li Y and Zhang L. J Biol Chem. 2003; 278:34395-34402). This domain contains a gene dimorphism, which encodes ITGAM with either a threonine (T) or a methionine (M) at amino acid position 425 (Frenzel H, et al. Immunogenetics. 2002; 53:835-842). The functional consequences of this SNP remains unknown but could modify the C3/ITGAM interaction. We have formulated the hypothesis that C3-80-RG and/or ITGAM-425-MT dimorphisms may influence cancer progression, including in subjects undergoing therapy, in this case with rituximab. Genotyping of C3-80-RG and ITGAM-425-MT were therefore performed on patients with previously untreated FL who had received rituximab alone. C1qA-70_{AG} (Racila D M, et al. Lupus. 2003; 12:124-132) and ITGAM-1130-PS (Frenzel H, et al. 2002) were also determined as controls since C1qA-70_{AG} polymorphism has been reported to be associated with progression free survival after rituximab treatment (Racila D M, et al. Blood. 2005; 106) and ITGAM-1130-PS polymorphism is localized outside of the interaction site between ITGAM and C3.

In the present disclosure, the inventors have genotyped C3 and ITGAM in a population of untreated FL patients receiving rituximab alone. This well-defined population has been extensively described and long-term outcome has been recently reported (Colombat P, et al. Blood 2001; 97:101-106; Colombat P, et al. Blood 2006; 108:486a). In the present disclosure, the inventors demonstrate that homozygous C3-80G patients have a lower probability to respond to rituximab compared to C3-80R carriers and that homozygous ITGAM-425M patients have a significant better progression free survival (PFS) compared to heterozygous ITGAM-MT patients. There is also a trend for a better overall survival (OS) for homozygous ITGAM-425M patients. In multivariate analysis, C3-80RG and ITGAM-245MT polymorphisms were the only factors influencing significantly response to rituximab and PFS, respectively, compared to C1q-70AG and ITGAM-1130PS control polymorphisms.

The present finding can be reconciled with observations that C3−/− and ITGAM−/− mice had partially abrogated antibody effects in a model of ADCC, whereby the CR3-ADCC mechanism consequently would have a direct effect on a subject's response to therapeutic antibody treatment (Imai M, et al. Cancer Res. 2005; 65:10562-10568; Van Spriel A B, et al. Blood. 2003; 101:253-258). The polymorphisms affecting the C3-ITGAM axis are therefore believed to affect a cooperative interaction with Fey receptors, in turn affecting for example activation of immune effector cells, and ADCC.

Typically, therapeutic antibodies will be directed to deplete (lead to the elimination of) target cells bearing a target antigen recognized by the therapeutic antibody (e.g. tumor cells), and preferably these antibodies will have the ability to induce ADCC of target cells. Typically, these antibodies will have constant regions of the G1 or G3 subtype, which bind Fc receptors and direct effector cells to lyse target, e.g. tumor, cells, although other subtypes (e.g. IgG2, IgG4) may retain effector function or Fc receptor binding ability, or may be modified (e.g. amino acid insertions, deletions or substitution, modifications to glycosylation such as hypofusocylation) to increase effector function Fc receptor binding ability. It will be appreciated that in any of the embodiments herein, an antigen-binding protein can be used in the same way as a therapeutic antibody in the context of the invention, particularly where such antigen-binding protein is directed to deplete target cells bearing a target antigen recognized by the therapeutic, and preferably the antigen-binding protein has the ability to induce. ADCC of target cells, and/or where the antigen-binding protein comprise an Fc portion.

Accordingly, the present disclosure demonstrates an association between the ITGAM and C3 genotypes and cancer progression, including clinical and molecular responses to therapy. The invention thus provides markers that can be used to monitor, evaluate or select a subject's cancer progression. This invention thus introduces new pharmacogenetical approaches in the management of subjects with malignancies, particularly B-cell hyperproliferative disorders.

### DEFINITIONS

A “genotype” is the genetic constitution of an individual (or group of individuals) at one or more genetic loci. Genotype is defined by the allele(s) of one or more known loci of the individual, typically, the compilation of alleles inherited from its parents.

A “polymorphism” is a locus that is variable; that is, within a population, the nucleotide sequence at a polymorphism has more than one version or allele. The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population.

An allele “positively” correlates with a trait when it is linked to it and when presence of the allele is an indictor that the trait or trait form will occur in an individual comprising the allele. An allele negatively correlates with a trait when it is linked to it and when presence of the allele is an indicator that a trait or trait form will not occur in an individual comprising the allele.

A marker polymorphism or allele is “correlated” or “associated” with a specified phenotype (e.g., increased response to a therapeutic antibody, etc.) when it can be statistically linked (positively or negatively) to the phenotype. That is, the specified polymorphism occurs more commonly in a case population (e.g., subjects having a greater antitumor response to treatment) than in a control population (e.g., subjects having a lower antitumor response to treatment).

A “favorable allele” is an allele at a particular locus that positively correlates with a desirable phenotype, e.g., greater survival, greater antitumor response

An “unfavorable allele” is an allele at a particular locus that negatively correlates with a desirable phenotype, or that correlates positively with an undesirable phenotype, e.g., lower survival, lower antitumor response.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles).

“Treatment regimen” as used herein, refers to treatment with a molecule alone, or in combination with another molecule. A treatment regimen also refers to dose amount, the frequency of dosing and the number of times a molecule, or combination of molecules, is administered

The term “biological sample” as used herein includes but is not limited to a biological fluid (for example serum, lymph, blood), cell sample or tissue sample (for example bone marrow).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). Heavy and light chains each contain a C-terminal constant region, common to all antibodies of a particular isotype, and an N-terminal variable region that confers binding specificity to the antibody. The term “antibody,” as used herein, refers to monoclonal antibodies regardless of their source or method of production, including, e.g., monospecific, polyspecific (e.g., bispecific), humanized, fully human, chimeric, recombinant, hybrid, mutated, and CDR grafted antibodies. It also includes portions of antibody molecules, such as scFv's, so long as such molecules are linked to an Fc region of an immunoglobulin. The term “polyclonal antibody,” as used herein, refers to recombinantly produced polyclonal antibodies. Polycolonal antibodies may be used in the methods and compositions of the invention similarly to other antibodies as described herein. Methods of making antibodies of these various types are well known and are described in, e.g., Antibody Engineering by Borrebaeck (editor), Oxford University Press, 2nd ed., 1995; Antibody Engineering: Methods and Protocols (Methods in Molecular Biology) by Lo (ed.), Humana Press, 2003; and Antibody Engineering (Springer Lab Manuals) by Kontermann et al. (eds.), Springer; 1st ed., 2001.

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, Md.).

The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, neutrophils, and eosinophils.

According to the invention, the ITGAM gene and C3 genes respectively refer to any nucleic acid molecule encoding an ITGAM or C3 polypeptide in a subject. This term includes, in particular, genomic DNA, cDNA, RNA (pre-mRNA, messenger RNA, etc.), etc. or any synthetic nucleic acid comprising all or part of the sequence thereof. Synthetic nucleic acid includes cDNA, prepared from RNAs, and containing at least a portion of a sequence of the ITGAM or C3 genomic DNA as for example one or more introns or a portion containing one or more mutations. Most preferably, the term ITGAM or C3 gene refers to genomic DNA, cDNA or mRNA, typically genomic DNA or mRNA. The ITGAM or C3 genes are preferably a human ITGAM or C3 gene or nucleic acid, i.e., comprises the sequence of a nucleic acid encoding all or part of an ITGAM or C3 polypeptide having the sequence of a human ITGAM or C3 polypeptide. Such nucleic acids can be isolated or prepared according to known techniques. For instance, they may be isolated from gene libraries or banks, by hybridization techniques. They can also be genetically or chemically synthesized. Within the context of this invention when referring to a gene or nucleic acid, a portion or part means at least 3 nucleotides (e.g., a codon), preferably at least 9 nucleotides, even more preferably at least 15 nucleotides, and can contain as much as 1000 nucleotides. Such a portion can be obtained by any technique well known in the art, e.g., enzymatic and/or chemical cleavage, chemical synthesis or a combination thereof.

Corbi et al. (1998) J. Biol. Chem. 263:12403-11 reported the complete amino acid sequence of ITGAM as deduced from cDNA for the human alpha subunit. The protein consists of 1,136 amino acids with a long amino-terminal extracytoplasmic domain, a 26-amino acid hydrophobic transmembrane segment, and a 19-carboxyl-terminal cytoplasmic domain. The sequence of a wild type ITGAM gene (cDNA) is represented in SEQ ID NO 1 (see also Genbank accession Number NM_{—}000632 for cDNA sequence). The amino acid sequence of human ITGAM is represented SEQ ID NO 2, having 1,152 amino acids and including a 16 amino acid signal peptide, as described in UniProtKB/Swiss-Prot accession number P11215 and Genbank accession number NP_{—}000623. Amino acid position 425 of ITGAM is numbered from residue 1 of the mature protein. It corresponds to residue 441 of the pre-protein having a signal peptide.

De Bruijn et al. (1985) P.N.A.S. USA 82 (3), 708-712 (1985) reported the amino acid sequence of human C3. The consensus sequence of a wild type C3 gene is represented in SEQ ID NO 3 (see also Genbank accession Number NM_{—}000064 for cDNA sequence). The amino acid sequence of human C3 is represented in SEQ ID NO 4, having a 1,663 amino acids including a 22 amino acid signal peptide. The mature C3 protein corresponds to amino acids 23 to 1663 in SEQ ID NO 4. Human C3 protein is also described in UniProtKB/Swiss-Prot accession number P01024 and Genbank accession number NP_{—}000055. Amino acid position 80 is numbered from residue 1 of the mature protein. It corresponds to residue 102 of the pre-protein having a signal peptide.

### Determining ITGAM and C3 Genotypes

Determining ITGAM or C3 genotype of a subject will generally involve obtaining from the subject a biological sample which comprises nucleic acids or proteins. The sample obtained from the host is assayed in vitro to determine the genotype of the host or subject from which the sample was obtained with respect to the ITGAM or C3 polymorphism. Optionally, the genotype of a subject with respect to both ITGAM and C3 polymorphisms can be assayed. Optionally, as further described below, the genotype of a subject with respect to at least one or more further non-ITGAM, non-C3 polymorphism(s) is assayed.

Preferably, determining the ITGAM genotype will involve determining the ITGAM-425 genotype of a subject comprises, where the amino acid residue at position 425 of ITGAM (or corresponding codon in the ITGAM gene) is determined. The method will comprise determining whether a methionine (M) or a threonine (T) is present at position 425, and preferably, whether a subject is heterozygous or homozygous for a threonine or methionine at position 425. The sequence of a portion of an ITGAM gene encoding amino acid position 425 is represented below, for sake of clarity. Nucleotide position 1419 to 1421 in the cDNA sequence of SEQ ID NO 1 corresponds to amino acid 425.

| | | |
|---|---|---|
| cDNA | 1401 1411 1421 1431 1440 | |
| 425M allele | ttcaggcaga acactggcat gtgggagtcc aacgctaatgtc | SEQ ID 5 |
| | F R Q N T G M W E S N A N V | SEQ ID 6 |
| 425T allele | ttcaggcaga acactggcac gtgggagtcc aacgctaatgtc | SEQ ID 7 |
| | F R Q N T G T W E S N A N V | SEQ ID 8 |

Preferably, determining the C3 genotype will involve determining the C3-80 genotype of a subject comprises, where the amino acid residue at position 80 of C3 (or corresponding codon in the C3 gene) is determined. The method will comprise determining whether an arginine (R) or a glycine (G) is present at position 80, and preferably, whether a subject is heterozygous or homozygous for an arginine or a glycine at position 80. The sequence of a portion of a C3 gene encoding amino acid position 80 is represented below, for sake of clarity. Nucleotide position 364 to 366 in the cDNA sequence of SEQ ID NO 3 corresponds to amino acid 80.

| | | |
|---|---|---|
| cDNA | 351 361 371 381 | |
| 80R allele | ttcaagtcagaaaaggggcg caacaagttcgtgaccgtgcag | SEQ ID 9 |
| | F K S E K G R N K F V T V Q | SEQ ID 10 |
| 80G allele | ttcaagtcagaaaagggggg caacaagttcgtgaccgtgcag | SEQ ID 11 |
| | F K S E K G G N K F V T V Q | SEQ ID 12 |

As indicated above, the invention comprises a method of determining in vitro the ITGAM-425 or C3-80 genotype of said subject. This more particularly comprises determining the nature of amino acid residue present (or encoded) at position 425 of the ITGAM polypeptide or position 80 of the C3 polypeptide.

Any convenient protocol for assaying a sample for the above ITGAM or C3 polymorphisms may be employed in the subject methods. In certain embodiments, the polymorphism will be detected at the protein level, e.g., by assaying for a polymorphic protein). Thus, determining the ITGAM or C3 genotype of said subject encompasses determining the nature of amino acid residue present (or encoded) at position 425 of the ITGAM polypeptide or position 80 of the C3 polypeptide. In other embodiments, the polymorphism will be detected at the nucleic acid level (e.g., by assaying for the presence of nucleic acid polymorphism, e.g., a nucleotide polymorphism that cause expression of the polymorphic protein.

For example, polynucleotide samples derived from (e.g., obtained from) a subject may be employed. Any biological sample that comprises a polynucleotide from the subject is suitable for use in the methods herein. The biological sample may be processed so as to isolate the polynucleotide. Alternatively, whole cells or other biological samples may be used without isolation of the polynucleotides contained therein. Detection of a target polymorphism in a polynucleotide sample derived from a subject can be accomplished by any means known in the art, including, but not limited to, amplification of a sequence with specific primers; determination of the nucleotide sequence of the polynucleotide sample; hybridization analysis; single strand conformational polymorphism analysis; restriction fragment length polymorphism analysis; denaturing gradient gel electrophoresis; mismatch cleavage detection; and the like. Detection of a target polymorphism can also be accomplished by detecting an alteration in the level of a mRNA transcript of the gene; aberrant modification of the corresponding gene, e.g., an aberrant methylation pattern; the presence of a non-wild-type splicing pattern of the corresponding mRNA; an alteration in the level of the corresponding polypeptide; and/or an alteration in corresponding polypeptide activity.

In an exemplary embodiment, the step of determining the amino acid residue at position 425 of ITGAM comprises a step of sequencing the ITGAM gene or RNA or a portion thereof comprising the nucleotides encoding amino acid residue 425 of the ITGAM gene. Determining amino acid residue at position 80 of C3 comprises a step of sequencing the C3 gene or RNA or a portion thereof comprising the nucleotides encoding amino acid residue 80 of the C3 gene.

In another exemplary embodiment, the step of determining the amino acid residue at position 425 of ITGAM comprises a step of amplifying the ITGAM gene or RNA or a portion thereof comprising the nucleotides encoding amino acid residue 425. Determining the amino acid residue at position 80 of C3 comprises a step of amplifying the C3 gene or RNA or a portion thereof comprising the nucleotides encoding amino acid residue 80. Amplification may be performed by polymerase chain reaction (PCR), such as simple PCR, RT-PCR or nested PCR, for instance, using conventional methods and primers.

In this regard, amplification primers for use in this invention more preferably contain less than about 50 nucleotides even more preferably less than 30 nucleotides, typically less than about 25 or 20 nucleotides. Also, preferred primers usually contain at least 5, preferably at least 8 nucleotides, to ensure specificity. The sequence of the primer can be prepared based on the sequence of the ITGAM or C3 genes, for example to allow full complementarity therewith. The probe may be labeled using any known techniques such as radioactivity, fluorescence, enzymatic, chemical, etc. This labeling can use for example Phosphorus32, biotin (16-dUTP), digoxygenin (11-dUTP). It should be understood that the present invention shall not be bound or limited by particular detection or labeling techniques. The primers may further comprise restriction sites to introduce allele-specific restriction sites in the amplified nucleic acids, as disclosed below.

Specific examples of such amplification primers are, for instance, SEQ ID NO: 13-16.

It will be appreciated that other primers can be designed, for example based on any fragment of the ITGAM or C3 gene, for use in the amplification step and especially a pair of primers comprising a forward sequence and a reverse sequence wherein said primers of said pair hybridize with a region of an ITGAM or C3 gene and allow amplification of at least a portion of the ITGAM or C3 gene containing codons encoding amino acid residue 425 or 80, respectively. In a preferred embodiment, each pair of primers comprises at least one primer that is complementary, and overlaps with codons encoding amino acid residue 425 or 80, respectively, permitting the discrimination between 425M and 425T alleles or 80R and 80G alleles. The amplification conditions may also be adjusted by the skilled person, based on common general knowledge and the guidance contained in the specification.

In a particular embodiment, the method of the present invention thus comprises a PCR amplification of a portion of the ITGAM or C3 mRNA or gDNA with specific oligonucleotide primers, in the cell or in the biological sample, said portion comprising the codon corresponding to amino acid position 425 of the ITGAM protein or position 80 of the C3 protein, and a direct or indirect analysis of PCR products, e.g., by electrophoresis, particularly Denaturing Gel Gradient Electrophoresis (DGGE).

In another embodiment, determining amino acid residue at position 425 of ITGAM or position 80 of C3 comprises a step of allele-specific restriction enzyme digestion. This can be done by using restriction enzymes that cleave the coding sequence of a particular allele (e.g., the 425M allele for ITGAM) and that do not cleave the other allele (e.g., the 425T allele, or vice versa). Where such allele-specific restriction enzyme sites are not present naturally in the sequence, they may be introduced therein artificially, by amplifying the nucleic acid with allele-specific amplification primers containing such a site in their sequence. Upon amplification, determining the presence of an allele may be carried out by analyzing the digestion products, for instance by electrophoresis. This technique also permits the identification of subjects that are homozygous or heterozygous for the selected allele. Examples of allele-specific amplification primers are disclosed in SEQ ID NOS 13-16.

In a further particular embodiment, determining amino acid residue at position 425 of ITGAM or position 80 of C3 comprises a step of hybridization of the ITGAM or C3 gene or RNA or a portion thereof comprising the nucleotides encoding amino acid residue 425 for ITGAM or amino acid residue 80 for C3, with a nucleic acid probe specific for the genotype methionine or threonine for ITGAM, or arginine or glycine for C3, and determining the presence or absence of hybrids.

It should be understood that the above methods can be used either alone or in various combinations. Furthermore, other techniques known to the skilled person may be used as well to determine the ITGAM-425 or C3-80 genotype, such as any method employing amplification (e.g. PCR), specific primers, specific probes, migration, etc., typically quantitative RT-PCR, LCR (Ligase Chain Reaction), TMA (Transcription Mediated Amplification), PCE (an enzyme amplified immunoassay) and bDNA (branched DNA signal amplification) assays.

In a preferred embodiment of this invention, determining amino acid residue at position 425 of ITGAM comprises:
obtaining genomic DNA from a biological sample,
amplifying the ITGAM gene or a portion thereof comprising the nucleotides encoding amino acid residue 425, and
determining amino acid residue at position 425 of said ITGAM gene.

Amplification can be accomplished with any specific technique such as PCR, including nested PCR, using specific primers as described above. In a most preferred embodiment, determining amino acid residue at position 425 is performed by allele-specific restriction enzyme digestion. In that case, the method comprises:
obtaining genomic DNA from a biological sample,
amplifying the ITGAM gene or a portion thereof comprising the nucleotides encoding amino acid residue 425,
introducing an allele-specific restriction site,
digesting the nucleic acids with the enzyme specific for said restriction site and,
analysing the digestion products, i.e., by electrophoresis, the presence of digestion products being indicative of the presence of the allele.

The methods can be carried out in the same way determine the amino acid residue at position 80 of C3.

In an other particular embodiment, the genotype is determined by a method involving extracting total (or messenger) RNA from cell or biological sample or biological fluid in vitro or ex vivo, optionally cDNA synthesis, (PCR) amplification with ITGAM-specific or C3-specific oligonucleotide primers, and analysis of PCR products.

The method of this invention may also comprise determining amino acid residue at position 425 of ITGAM directly by sequencing the ITGAM polypeptide or a portion thereof comprising amino acid residue 425 or by using reagents specific for an allele of interest of the ITGAM polypeptide. Determining amino acid residue at position 80 of C3 may comprise directly sequencing the C3 polypeptide or a portion thereof comprising amino acid residue 80 or by using reagents specific for each of the allele of interest of the C3 polypeptide. A variety of methods for detecting polypeptides can be employed and include, for example, any protein sequencing method following extraction of proteins from a sample (e.g. Edman type), immunohistochemical analysis, immunoprecipitation, Western blot analysis, molecular binding assays, ELISA, EIA, RIA, ELIFA, fluorescence activated cell sorting (FACS), mass spectroscopy, protein microarray, and the like. In some embodiments, an ITGAM or C3 polypeptide in a biological sample is detected by (a) contacting the sample with an ITGAM or C3 binding agent, such as an antibody, a fragment thereof, or a protein (such as a recombinant protein) containing an ITGAM or C3 binding region; and (b) detecting the ITGAM or C3 binding agent—ITGAM or C3 polypeptide complex in the sample. Several methods will use an affinity reagent specific for an ITGAM-425 or C3-80 polypeptide, more preferably any antibody or fragment or derivative thereof. In a particular embodiment, the ITGAM-425 or C3-80 polypeptide is detected with an anti-ITGAM-425 or anti-C3-80 antibody (e.g. a monoclonal antibody or a fragment thereof) that discriminates between ITGAM-425M and ITGAM-425-T or between C3-80-G and or C3-80-R, respectively. The antibody (or affinity reagent) may be labelled by any suitable method (radioactivity, fluorescence, enzymatic, chemical, etc.). Alternatively, ITGAM-425M antibody immune complexes may be revealed (and/or quantified) using a second reagent (e.g., antibody), labelled, that binds to the anti-ITGAM-425-M antibody, for instance. ITGAM or C3 polypeptides also can be detected by mass spectrometry assays for example coupled to immunaffinity assays, the use of matrix-assisted laser desorption/ionization time-of-flight (MALDI-TOF) mass mapping and liquid chromatography/quadrupole time-of-flight electrospray ionization tandem mass spectrometry (LC/Q-TOF-ESI-MS/MS) sequence tag of extracted proteins separated by two-dimensional polyacrylamide gel electrophoresis (2D-PAGE) (Kiernan et al., Anal. Biochem., 301: 49-56, 2002; Poutanen et al., Mass Spectrom., 15: 1685-1692, 2001).

The above methods are based on the genotyping of ITGAM-425 or C3-80 in a biological sample of the subject. The biological sample may be any sample containing an ITGAM or C3 gene or corresponding polypeptide, particularly blood, bone marrow, lymph node, epithelial cells or more generally any somatic cell from a subject. Furthermore, because the ITGAM and C3 genes are generally present on or within the cells, tissues or fluids mentioned above, the method of this invention usually uses a sample treated to render the gene or polypeptide available for detection or analysis. Treatment may comprise any conventional fixation techniques, cell lysis (mechanical or chemical or physical), or any other conventional method used in immunohistology or biology, for instance.

### Correlating Genotype to Cancer Prognosis

Generally, so long as information about a subject's ITGAM and C3 genotype (e.g. ITGAM-425 or C3-80 genotype) is available (e.g. retrieved from a database, in a patient record), the subject's genotype can be correlated to a prediction or indication concerning a subject's cancer prognosis. As discussed, the method may include detecting, in the organism or biological sample, the allele present at a polymorphism or a locus closely linked thereto, the polymorphism being in an ITGAM or C3 gene, wherein the polymorphism is associated with cancer prognosis, including cancer prognosis in a patient undergoing treatment with an anti-cancer therapy. Thus, in any of the embodiments herein, the methods further include correlating said polymorphism, genotype or locus to a cancer prognosis.

With respect to ITGAM, a subject having a methionine at amino acid residue position 425 of ITGAM will be designated herein as having a “favourable allele” or a “favorable cancer prognostic”, as this subject will have an improved cancer prognosis compared to another subject (e.g. a subject having an unfavorable allele). A subject having a threonine at amino acid residue position 425 of ITGAM will be designated herein as having an “unfavourable allele” or an “unfavorable cancer prognostic”, as this subject will have a less favourable cancer prognosis compared to a subject having a favorable allele. Preferably, heterozygosity or homozygosity for a threonine (T) at position 425 is indicative of an unfavorable cancer prognostic, and homozygosity for a methionine is indicative of a favorable cancer prognostic.

With respect to C3, a subject having a arginine (R) at amino acid residue position 80 of C3 will be designated herein as having a “favourable allele” or a “favorable cancer prognostic”, as this subject will have an improved cancer prognosis compared to another subject (e.g. a subject having an unfavorable allele). A subject having a guanine (G) at amino acid residue position 80 of C3 will be designated herein as having an “unfavourable allele” or an “unfavorable cancer prognostic”, as this subject will have a less favourable cancer prognosis compared to a subject having a favorable allele. Preferably, homozygosity for a glycine at position 80 is indicative of an unfavourable cancer prognostic, and heterozygosity or homozygosity for an arginine is indicative of a favourable cancer prognostic.

“Cancer prognosis” generally refers to a forecast or prediction of the probable course or outcome of the cancer. As used herein, cancer prognosis includes but is not limited to the forecast or prediction of any one or more of the following: duration of survival of a subject susceptible to or diagnosed with a cancer, duration of recurrence-free survival, duration of progression free survival of a subject susceptible to or diagnosed with a cancer, response rate in a subject or in a group of subjects susceptible to or diagnosed with a cancer, duration of response in a subject or a group of subjects susceptible to or diagnosed with a cancer. As used herein, “prognostic for cancer” means providing a forecast or prediction of the probable course or outcome of the cancer. In some embodiments, “prognostic for cancer” comprises providing the forecast or prediction of (prognostic for) any one or more of the following: duration of survival of a subject susceptible to or diagnosed with a cancer, duration of recurrence-free survival, duration of progression free survival of a subject susceptible to or diagnosed with a cancer, response rate in a subject or group of subjects susceptible to or diagnosed with a cancer, duration of response in a subject or a group of subjects susceptible to or diagnosed with a cancer.

In one example, the prognosis defines outcome in the absence of anti-cancer therapy, or independently of anti-cancer therapy. Such outcome information, as further discussed below can be useful in selecting a treatment regimen for a subject. In most cases, however, the prognosis will be used to define outcome upon treatment of the cancer. Preferably the treatment comprises administration of a therapeutic antibody. Response rate is defined as the percentage of treated subjects who responded to a treatment. Duration of response is defined as the time from the initial response to treatment to disease progression. Time to disease progression is defined as the time from administration of treatment until disease progression. For example, the prognosis may be that a subject has a greater or lower likelihood to experience a particular duration of progression free survival (PFS) or duration overall survival (OS), or an objective response (OR) or complete response (CR). In some embodiments, in a subject receiving a treatment, duration of survival and duration of progression free survival are predicted.

In some embodiments, the prognosis defines outcome with a particular anti-cancer treatment regimen; for example, the prognosis may define outcome following treatment with a particular treatment regimen which is known to have at least some degree of efficacy as an anti-cancer therapy. The treatment regimen may comprise the administration of a sole anti-cancer therapy (i.e. monotherapy) or combination therapy. In some embodiments, the therapy comprises administration of a therapeutic antibody. The therapy may be for example in the presence of a particular type of adjuvant therapy, or in the absence of a particular type of adjuvant therapy (e.g. a therapy known to have toxicity, a chemotherapy, etc.).

For example, the prognosis may define outcome following treatment of a subject having a B-cell malignancy treated with an anti-CD20 antibody (e.g. rituximab), in the presence or absence of an adjuvant therapy, optionally wherein the adjuvant increases the efficacy of the antibody (e.g. has a synergistic effect), or wherein the adjuvant is a non-antibody anti-cancer agent having an additive effect or toxicity (e.g. chemotherapy). In another example, prognosis defines outcome following treatment a subject having a colon, breast, lung or other solid tumor with an antibody specific to a human EGF-like receptor family, an anti-HER-2 or HER-2/neu antibody, an anti-EGFR antibody, or an anti-IGR1R antibody. In another example, prognosis defines outcome following treatment a subject having a leukemia with an anti-CD20 (e.g. CLL), anti-CD52 or anti-CD33 antibody.

For example, correlating a subject's genotype to a cancer prognosis for a subject treated with a therapeutic antibody in the presence or absence of a particular adjuvant therapy will be useful for selecting the optimal therapeutic regimen for the subject. For example, the adjuvant therapy may be chemotherapy, where it would be advantageous to identify subjects with a favorable cancer prognostic who will have an increased response to the therapeutic antibody (e.g. a monotherapy or a combination of agents), and subjects with an unfavorable cancer prognostic who would have a decreased response to therapy (e.g. a monotherapy or a combination of agents), and would therefore benefit from an adjuvant therapy, e.g. chemotherapy.

Thus, a subject who is determined to have a favorable allele or genotype based on its ITGAM or C3 genotype will be expected to have a favourable cancer prognosis, e.g. greater duration of survival, greater duration of recurrence-free survival, greater duration of progression free survival of a subject susceptible to or diagnosed with a cancer, greater response rate. A subject who is determined to have an unfavorable allele or genotype based on its ITGAM or C3 genotype will be expected to have a less favourable cancer prognosis, e.g. lower duration of survival, lower duration of recurrence-free survival, lower duration of progression free survival of a subject susceptible to or diagnosed with a cancer, lower response rate.

In one aspect, the ITGAM genotype is used to assess a subject's survival, e.g. is likelihood to experience progressive disease, a particular duration of progression free survival (PFS) or overall survival (OS). In one aspect, the C3 genotype is used to assess a subject's short term response to therapy, e.g. objective response, objective response at least 1, 2, 3, 4, 5 or 6 months following treatment.

In certain embodiments, the genotype information is employed to give a refined probability determination as to whether a subject will or will not respond to a particular therapy. For example, an identification of the ITGAM-425M genotype and/or the C3-80R genotype may be employed to determine that the subject has at least a 70% chance, such as at least a 75% chance, including at least an 80% chance of responding to treatment, e.g., with rituximab. Likewise, an identification of the ITGAM-425T genotype and/or the C3-80G genotype may be employed to determine that the subject has less than 50% chance, such as a less than 45% chance, including a less than 40% chance of responding to treatment, e.g., with rituximab. In a preferred embodiment, the prognosis may be defined with respect to a particular treatment regimen and disease, where in the case of rituximab for the treatment of B cell lymphomas, rituximab is provided as weekly infusions of at a dose of 375 mg/m².

Correlating a subject's genotype to a cancer prognosis will take into account the nature of the cancer, the individual subject. When the prognosis defines outcome to a therapy, the nature of the particularly therapy and treatment regimen will be taken into account as well. As discussed herein, C3 and ITGAM are expressed on immune effector cells such as granulocytes, macrophages or NK cells. As such, the C3-ITGAM axis is believed to be indicative of a mechanism contributing to an individual's ability to mount an anti-cancer response.

More specifically, the methods of the present invention are utilized in the prognosis and treatment of a variety of cancers including, but not limited to, carcinoma, including that of the bladder, breast, colon, kidney, liver, lung, ovary, prostate, pancreas, stomach, cervix, thyroid and skin, including squamous cell carcinoma; hematopoietic tumors of lymphoid lineage, including leukemia, acute B or T lymphoblastic leukemia, chronic lymphocytic leukaemia, B-cell lymphoma (including FL, DLBCL, waldenstrom macroglobulinemia, lymphocytic, lymphoplasmocytoid, mantle cell and marginal zone lymphoma) T-cell lymphoma (including nodal and extra-nodal lymphoma), Hodgkin's lymphoma, hairy cell leukaemia, multiple myeloma; hematopoietic tumors of myeloid lineage, including acute leukaemia, chronic myeloproliferative disorders (including chronic myelogenous leukaemia, polycythemia vera, essential thrombocytemia, primaru melofibrosis, hypereosinophilic syndrome) and myelodysplasia; other tumors, including melanoma, seminoma, teratocarcinoma, neuroblastoma, PNET and glioma; tumors of the central and peripheral nervous system, including astrocytoma, neuroblastoma, glioma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma.

The term “therapeutic antibody” as used herein generally includes any antibody that has a mechanism of action that is directed to the depletion or elimination, of a target cell, e.g. a cell expressing the antigen toward which the antibody has specificity. A therapeutic antibody will typically comprise an Fc portion and will mediate a cytotoxic effect or cell lysis, particularly by antibody-dependant cell-mediated cytotoxicity (ADCC) toward a cell expressing the antigen for which the antibody binds via its antigen-binding domain (e.g. variable region, CDR regions). Such antibodies include antibodies that bind to Fey receptors present on cytotoxic effector cells (e.g. via their Fc portion), since CR3 and FcγRIIIA are believed to have a cooperative function. Binding of the antibody to a target cell results in killing of the target cell via ADCC, and where killing of the target cell(s) provides for a therapeutic effect in an individual. The therapeutic antibody may recruit monocytes, NK cells and granulocytes; the antibody may induce effector cell activity mediated via FcγR present on effector cells (e.g. FcγRIIIA on NK cells). Therapeutic antibodies may be designed to lead to elimination of target cells in a subject by immune effector cells, particularly effectors cells bearing FcγR and ITGAM proteins (e.g. NK cells). It will be appreciated that any polypeptide which comprises an antigen binding portion can be used in the same way as a therapeutic antibody in the methods of the invention, particularly an Fc fusion protein comprising an Fc portion and an antigen binding portion.

In the methods of the invention, the therapeutic antibodies are fully human, or otherwise contain the Fc domain of human antibodies, e.g., humanized or chimeric antibodies and Fc fusion molecules with a human Fc domain or a functional derivative thereof (e.g., a derivative that binds to one or more Fc receptors, e.g., FcγRIIIA). The derivatives include, for example, native sequences in which conservative substitutions were made and/or amino acids were deleted or inserted.

In preferred embodiments, the Fc portion of the therapeutic antibody is derived from human IgG1 or IgG3 since such antibodies typically are potent activators of ADCC. However, the invention can also be practiced with other classes of antibodies, including IgG, IgA, IgD, IgE and IgM, and isotypes, such as, e.g., IgG1, IgG2, IgG3, IgG4, IgA1, and IgA2. For example, human IgG4 has limited capacity to activate effector functions, but it still known to show some binding to FcγRIIIA and may therefore retain ability to induce ADCC or production of cytokines by Fcγ receptor-expressing cells; IgAs are potent activators of ADCC. Likewise, Fc portions of various subtype can be engineered to augment or reduce their complement or Fcγ receptor-binding properties.

The therapeutic antibody may be produced by a hybridoma or by recombinant cells engineered to express the desired variable and constant domains. The antibodies may be single chain antibodies or other antibody derivatives retaining the antigen specificity and the lower hinge region or a variant thereof. These may be polyfunctional antibodies, recombinant antibodies, ScFv, humanized antibodies, or variants thereof. Therapeutic antibodies are specific for surface antigens, e.g., membrane antigens. Examples of surface antigens and exemplary diseases contemplated herein include CD3 (e.g., non-Hodgkin's Lymphoma), CD4, CD5, CD6, CD8, CD14, CD15, CD16, CD19 (e.g., non-Hodgkin's Lymphoma), CD20, CD21, CD22, CD23, CD25, CD32B, CD30 (e.g., Hodgkin's Disease), CD33, CD37, CD38, CD40, CD40L, CD44 and its splice variant CD44v6CD46, CD52, CD54, CD56, CD59, CD70, CD74, CD79, CD80, CD122, CD126, CD133, CD138, CD137 and CD152. In some embodiments the antibodies can be directed to an oncogene, an oncogene product, a necrosis antigen, IL-2 receptor, TAC, TRAIL-R1, GD3 ganglioside or TRAIL-R2. Other targets include: (CTLA-4), CD200, CD317 (HM1.24), human leukocyte antigen (HLA)-DR, Flt3, CCR4, BR3/Blys3R, EpCAM, MUC1, MCAM/MUC18, podoplanin, CEA (carcinoembryonic antigen), PDGFR, GD2, GD3, GM2 and GM3 gangliosides, LeY, PSMA (prostate specific membrane antigen), PSCA (prostate stem cell antigen), A33, CAIX/MN, TRAIL-R1 and TRAIL-R2, HMW-MMA (human high molecular weight melanoma associated antigen), BCMA (B-cell maturation antigen), FRA (folate receptor α)/gp38, tenascin, phosphatidylserine, GFAP (glial fibrillary acidic protein), AMVB1, Tn-antigen, ICAM1, IL6-R, HGFR, CRIPTO antigens (e.g. CRIPTO-1, CRIPTO-3), a member of FGF receptor family including FGFR1 and FGFR3.

Therapeutic antibodies may be specific for any tumor antigens including for example MAGE, MART-1/Melan-A, gp100, dipeptidyl peptidase IV (DPPIV), adenosine deaminase-binding protein (ADAbp), cyclophilin b, carcinoembryonic antigen (CEA) and its immunogenic epitopes CAP-1 and CAP-2, etv6, aml1, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A1, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, VEGF, VEGF receptors, A-Raf, B-Raf, C-Raf, Raf-1, HSP70, HSP90, PDGF, TGF-alpha, EGF, EGF receptor (e.g. antibodies IMC-11F8 or Cetuximab (ERBITUX®) Imclone Systems Inc.), IGF-1 receptor, a member of the human EGF-like receptor family such as HER-2/neu (e.g. antibody trastuzumab (Herceptin®), Gen entech), HER-3, HER-4 or a heterodimeric receptor comprised of at least one HER subunit, gastrin releasing peptide receptor antigen, Muc-1, CA125, integrins (e.g. αvβ3 integrins, α5β1 integrins, αIIbβ3-integrins), PDGF beta receptor, Src, VE-cadherin, IL-8, hCG, IL-6, IL-6 receptor, p21ras, RCAS1, α-fetoprotein, E-cadherin, α-catenin, β-catenin and γ-catenin, p120ctn, PRAME, NY-ESO-1, cdc27, adenomatous polyposis coli protein (APC), fodrin, Connexin 37, Ig-idiotype, p15, gp75, p97, GM2 and GD2 gangliosides, viral products such as human papillomavirus proteins, Smad family of tumor antigens, imp-1, PIA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2, or any additional protein target set forth in http://oncologyknowledgebase.com/oksite/TargetedTherapeutics/TTOExhibit2.pdf and http://oncologyknowledgebase.com/oksite/TargetedTherapeutics/TTOExhibit3.pdf, the disclosures of which are herein incorporated by reference. This list is not meant to be limiting.

### Treatment

Once a subject is identified as having a favourable or unfavorable cancer prognostic, e.g. the subject has a favourable or unfavorable allele for ITGAM or C3, steps can be taken to determine an appropriate therapeutic regimen for the subject, or for example whether to include the subject in a study (e.g. selecting a subject or biological sample from a subject for analysis, selecting a subject for inclusion in a clinical trial). Based on a subject's ITGAM or C3 genotype it will be possible to select from therapeutic regimens involving monotherapy, combination therapies (e.g. treatment with an agent with or without an adjuvant), the intensity and nature of the therapeutic regimen (e.g. dosage, administration schedule), or to select between particular agents, e.g. to select a chemotherapeutic agent or an antibody agent having increased potency over another chemotherapeutic or antibody agent.

Adjuvant therapy can generally comprise adding, to a treatment with a first therapy or agent, any one or more treatments that have the potential to be additive or synergistic with a treatment. For example, when a therapeutic antibody is selected to treat a subject, an adjuvant therapy may comprise an agent other than the particular therapeutic antibody; for example, the agent may be any agent that has a mechanism of action different from the therapeutic antibody, including for example a second therapeutic antibody that is specific for a different antigen that the therapeutic antibody, an non-antibody immunotherapeutic agent, a small molecule compound that acts on a different biological target, a chemotherapeutic agent, an agent that enhances the efficacy of the therapeutic antibody, etc. In one embodiment, the adjuvant is an agent that is known to have an anti-cancer activity when administered without a therapeutic antibody, or on its own; in another embodiment, the adjuvant is an agent is a compound that can modulate a subject's immune system and that has synergistic activity with a therapeutic antibody. Such immune modulating compounds may include include for example, cytokines, interleukins, PAMPs (for “pathogen-associated molecular patterns”), CpG-containing oligonucleotides, selected chemotherapeutic agents, beta-glucan compositions report that interleukin-15 (IL-15) and CpG oligodeoxynucleotides A-Class enhance rituximab-mediated ADCC against B-cell lymphoma (Moga et al. Exp Hematol. (2008) 36(1):69-77). Van Ojik et al. Cancer Res. (2003) 63(17):5595-600 report that other classes of CpG ODN increase the potency of rituximab; Cheung N K and Modak S, (2002) Clin. Cancer Res. 8:1217-1223 report that beta-glucan (polymers of glucose, e.g. beta-1,3 glucans, beta-1,3/1,6-glucan, glucan from yeast, oats, barley, seaweed, mushrooms) synergize with antiganglioside antibodies; Zitvogel L et al., (2008) Nat. Rev. Immunol. 8: 59-73 reviews immunological aspects of conventional cancer treatments, all of which treatments are incoroporated herein by reference.).

Selecting among therapies can involve selecting a chemotherapeutic agent or an antibody agent having increased potency over another chemotherapeutic or antibody agent. For example, an antibody agent having increased potency can be an antibody comprising an Fc portion that, compared to a naturally occurring human Fc portion, is modified to have increased binding to Fcγ receptor(s) (e.g. FcγRIIIa on effector (e.g. NK) cells). Typical modifications include modified human IgG1 constant regions comprising at least one amino acid modification (e.g. substitution, deletions, insertions), and/or altered types of glycosylation, e.g., hypofucosylation. Certain altered glycosylation patterns in constant regions have been demonstrated to increase the ADCC ability of antibodies.

Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 06/133148; WO 03/035835; WO 99/54342 80, each of which is incorporated herein by reference in its entirety. Generally, such antibodies with altered glycosylation have a particular N-glycan structure that produces certain desirable properties, including but not limited to, enhanced ADCC and effector cell receptor binding activity when compared to non-modified antibodies or antibodies having a naturally occurring constant region and produced by murine myeloma NSO and Chinese Hamster Ovary (CHO) cells (Chu and Robinson, Current Opinion Biotechnol. 2001, 12: 180-7) or other mammalian host cell lines commonly used to produce recombinant therapeutic antibodies. Monoclonal antibodies produced in mammalian host cells contain an N-linked glycosylation site at Asn297 of each heavy chain. Glycans on antibodies are typically complex biatennary structures with very low or no bisecting N-acetylglucosamine (bisecting GlcNAc) and high levels of core fucosylation. Glycan temini contain very low or no terminal sialic acid and variable amounts of galactose. For a review of glycosylation on antibody function, see, e.g., Wright & Morrison, Trend Biotechnol. 15:26-31 (1997). The important carbohydrate structures contributing to antibody activity are believed to be the fucose residues attached via alpha-1,6 linkage to the innermost N-acetylglucosamine (GlacNAc) residues of the Fc region N-linked oligosaccharides (Shields et al., 2002). FcγR binding requires the presence of oligosaccharides covalently attached at the conserved Asn297 in the Fc region. Non-fucosylated structures have recently been associated with dramatically increased in vitro ADCC activity. Historically, antibodies produced in CHO cells contain about 2 to 6% in the population that are nonfucosylated. YB2/0 (rat myeloma) and Lec13 cell line (a lectin mutant of CHO line which has a deficient GDP-mannose 4,6-dehydratase leading to the deficiency of GDP-fucose or GDP sugar intermediates that are the substrate of alpha6-fucosyltransferase have been reported to produce antibodies with 78 to 98% non-fucosylated species. In other examples, RNA interference (RNAi) or knock-out techniques can be employed to engineer cells to either decrease the FUT8 mRNA transcript levels or knock out gene expression entirely, and such antibodies have been reported to contain up to 70% non-fucosylated glycan. In other examples, a cell line producing an antibody can be treated with a glycosylation inhibitor; Zhou et al. Biotech. and Bioengin. 99: 652-665 (2008) described treatment of CHO cells with the alpha-mannosidase I inhibitor, kifunensine, resulting in the production of antibodies with non-fucosylated oligomannose-type N-glucans. Thus, in one embodiment of the invention, a therapeutic antibody having increased potency will comprise a constant region comprising at least one amino acid alteration in the Fc region that improves antibody binding to FcγRIIIa and/or ADCC. In another aspect, a therapeutic antibody having increased potency is hypofucosylated, e.g. wherein at least 20, 30, 40, 50, 60, 75, 85 or 95% of the antibodies in the composition have a constant region comprising a core carbohydrate structure which lacks fucose.

### Favorable Cancer Prognostics

Subjects having a favorable cancer prognostic are expected to have a better response to a therapy. It will be therefore be advantageous to adapt the treatment regimen for these subjects by either administering a standard anti-cancer agent or therapeutic regimen, or by decreasing the intensity of the therapeutic regimen so as to decrease side effects, cost, etc.

Particularly where a therapy (e.g. administered according to a standard treatment regimen) has been tested in a population without distinguishing for ITGAM or C3 genotypes, it may be advantageous to treat a subject having a favorable cancer prognostic with said therapy and/or standard regimen, e.g. the regulatory agency-approved or commonly used regimen. Such regimen may involve a monotherapy or a combination therapy (e.g. maintenance therapy). For example, rituximab has been approved as a monotherapy for treatment of relapsed or refractory low-grade follicular NHL in a population without distinguishing for ITGAM or C3 genotypes as shown herein, that as such is expected to include a majority of high responders for C3 and a majority of high responders for ITGAM, since approximately 91% (63%+28%) of subjects genotyped herein were C3 high responders and 67% of subjects were ITGAM high responders. Optionally, such a treatment will exclude treatment with a particular adjuvant therapy. For example, high doses chemotherapy having toxic side effects may be avoided.

For example, the methods may comprise predicting a cancer prognosis for a subject based on C3 or ITGAM genotype, where the prognosis predicts response to a therapeutic antibody, and if the subject has a favourable cancer prognostic, selecting a therapeutic antibody and optionally treating the subject with the therapeutic antibody. Optionally, the therapeutic antibody is administered in the absence of a particular adjuvant therapy, optionally wherein the adjuvant therapy has toxicity, or wherein the adjuvant is a chemotherapy. Optionally, the therapeutic antibody is administered as a monotherapy. Optionally, the therapeutic antibody is administered at lower dosage, frequency or for a lower duration that for subject having an unfavourable cancer prognostic or compared to a standard treatment regimen involving the therapeutic antibody.

More generally, the subject having a favorable allele can be treated with an adapted treatment regimen. For example, the subject can be treated with a treatment regimen that involves a lower dosage (e.g. lower than used in a subject having an unfavorable allele, or lower than a treatment regimen which does not distinguish between genotypes), less frequent administration, or shorter duration. In some aspects, a treatment regimen adapted to high responder may comprise removing, substituting or adapting an adjuvant therapy used in combination with the therapeutic antibody. For example, a subject having a favorable allele having cancer (e.g. a B cell lymphoma) can be treated with a therapy (e.g. an anti-CD20 antibody) but without one or more additional chemotherapeutic agent typically used to treat subjects, optionally as monotherapy (e.g. with a therapeutic antibody as monotherapy), or with an additional chemotherapeutic agent but in an adapted regimen (e.g. lower dosage).

Adapted treatment regimens for subjects having a favourable cancer prognosis may include for example any of the following:
i) a treatment regimen comprising a therapeutic agent administered at a standard dose and/or administration schedule recommended for subjects having the cancer;
ii) a treatment regimen comprising a therapeutic agent administered at a dose and/or administration schedule lower than for subjects having an unfavourable cancer prognostic or lower than that of the standard dose and/or administration schedule; and/or
iii) a treatment regimen comprising a therapeutic agent administered in the absence of a particular adjuvant, optionally wherein the adjuvant is a chemotherapeutic agent or an immune system modulating agent, optionally wherein the therapeutic agent is administered as sole anti-cancer agent, optionally wherein the therapeutic agent is administered at a standard or decreased dose and/or administration schedule recommended for subjects having the cancer, optionally wherein the therapeutic agent is an anti-cancer agent other than a chemotherapeutic agent, optionally wherein the therapeutic agent is an anti-CD20 antibody.

### Unfavorable Cancer Prognostics

In subjects having an unfavorable allele, the treatment regimen, e.g. a standard treatment regimen tested without distinguishing between ITGAM or C3 genotypes, may be adapted by modifying, optionally increasing the intensity of, a treatment regimen indicated for a particular cancer. Increasing the intensity of a therapeutic regimen that comprises a therapeutic antibody can involve for example administering a therapeutic agent at a higher dose or higher frequency of administration or for a longer duration, e.g. compared to a reference therapeutic regimen, or treating the subject in combination with an adjuvant therapy, administering the antibody with an adjuvant, or administering a therapeutic antibody having increased potency.

For example, the methods may comprise predicting a cancer prognosis for a subject based on C3 or ITGAM genotype, where the prognosis predicts response to a therapeutic antibody, and if the subject has an unfavourable cancer prognostic, selecting a therapeutic antibody and optionally treating the subject with the therapeutic antibody. Optionally the therapeutic antibody is an antibody having increased potency (e.g. hypofucosylated). Optionally, the therapeutic antibody is administered in the combination with a particular adjuvant therapy, optionally wherein the adjuvant therapy has toxicity (e.g. a chemotherapy), or wherein the adjuvant therapy increases the efficacy of the anti-cancer treatment (e.g. CpG oligonucleotides, cytokines, beta-glucans, immunomodulatory chemotherapeutic agents, etc.). The adjuvant therapy will typically have additive or preferably synergistic effects with the therapeutic antibody. Optionally, the therapeutic antibody is administered at higher dosage, frequency or for a longer duration that for subject having a favourable cancer prognostic or compared to a standard treatment regimen involving the therapeutic antibody. In another aspect, if a subject has unfavourable cancer prognostic with respect to response to a therapeutic antibody, the method may comprise selecting a therapeutic regimen that does not comprise said therapeutic antibody (e.g. a therapeutic regimen comprising an alternative therapeutic antibody, comprising chemotherapy, etc.), and optionally treating the subject with the therapeutic regimen.

In other aspects, where a particular therapy has been determined to be effective in the treatment of cancer in a population of subjects having an unfavourable cancer prognostic, such therapy can be used advantageously to treat such subjects. For example, the methods may comprise treating a subject having an unfavourable cancer prognostic with a therapy (e.g. a therapeutic regimen comprising a therapeutic antibody, optionally in combination with an adjuvant) effective in subjects having an unfavourable cancer prognostic based on C3 or ITGAM genotype. Optionally said therapy can be specially adapted to subjects having an unfavourable cancer prognostic based on C3 or ITGAM genotype

Adapted treatment regimens for unfavourable cancer prognostic may include for example any of the following:
i) a treatment regimen comprising a therapeutic agent administered at a standard dose and/or administration schedule recommended for subjects having the cancer;
ii) a treatment regimen comprising a therapeutic agent administered at a dose and/or administration schedule higher than for subjects having a favourable cancer prognostic or higher than that of the standard dose and/or administration schedule;
iii) a treatment regimen comprising a therapeutic agent administered in combination with an adjuvant, optionally wherein the adjuvant is a chemotherapeutic agent or immune system modulating agent, optionally wherein the therapeutic agent is administered at a standard or higher dose and administration schedule recommended for subjects having the cancer, optionally wherein the therapeutic agent is an anti-CD20 antibody; and/or
iv) a treatment regimen comprising a therapeutic antibody having increased potency, e.g. an antibody designed to have greater potency than an antibody containing a standard Fc portion, for example an antibody having an Fc portion that, compared to a naturally occurring human Fc portion, has increased binding to Fcγ receptor(s), is glycosylation modified, hypofucosylated or comprising an amino acid insertion, substitution or deletion.

In some embodiments, the prognostic methods are used to select and treat subjects having a B-cell hyperproliferative disorders, optionally a CD20-expressing disorder, and optionally further where the subjects are treated with a therapeutic antibody, optionally an anti-CD20 antibody. B-cell hyperproliferative disorders are those disorders that derive from cells in the B cell lineage, typically including hematopoietic progenitor cells expressing B lineage markers, pro-B cells, pre-B cells, B-cells and memory B cells; and that express markers typically found on such B lineage cells. Of particular interest are non-Hodgkin's lymphomas (NHLs), which are a heterogeneous group of lymphoproliferative malignancies with different patterns of behavior and responses to treatment. NHLs can be divided into 2 prognostic groups; the indolent lymphomas and the aggressive lymphomas. Indolent NHL types have a relatively good prognosis, with median survival as long as 10 years, but they usually are not curable in advanced clinical stages. The aggressive type of NHL has a shorter natural history. A number of these patients can be cured with intensive combination chemotherapy regimens, but there is a significant number of relapses, particularly in the first 2 years after therapy. Among the NHL are a variety of B-cell neoplasms, including precursor B-lymphoblastic leukemia/lymphoma; peripheral B-cell neoplasms, e.g. B-cell chronic lymphocytic leukemia; prolymphocytic leukemia; small lymphocytic lymphoma; mantle cell lymphoma; follicle center cell lymphoma; marginal zone B-cell lymphoma; splenic marginal zone lymphoma; hairy cell leukemia; diffuse large B-cell lymphoma; T-cell rich B-cell lymphoma, Burkitt's lymphoma; high-grade B-cell lymphoma, (Burkitt-like); etc. Follicular lymphoma comprises 70% of the indolent lymphomas reported in American and European clinical trials. Most patients with follicular lymphoma are over age 50 and present with widespread disease at diagnosis. Nodal involvement is most common, often accompanied by splenic and bone marrow disease. The vast majority of patients are diagnosed with advanced stage follicular lymphoma and are not cured with current therapeutic options, and the rate of relapse is fairly consistent over time, even in patients who have achieved complete responses to treatment. Subtypes include follicular small cleaved cell (grade 1) and follicular mixed small cleaved and large cell (grade 2). Another subtype of interest is follicular large cell (grade 3 or FLC) lymphoma which can be divided into grades 3a and 3b. Any of these disorders, subtypes, therapeutic settings or patients characteristics can be specified in any of the embodiments of the invention.

CD20 is a human B cell marker that is expressed during early pre-B cell development and remains until plasma cell differentiation. The CD20 molecule may regulate a step in the activation process that is required for cell cycle initiation and differentiation, and is usually expressed at very high levels on neoplastic B cells. Thus, the CD20 surface antigen can be targeted for treating B cell lymphomas. U.S. Pat. No. 5,736,137, herein incorporated by reference, describes the chimeric antibody “C2B8” that binds the CD20 antigen and its use to treat B-cell lymphoma (antibody is also known as Rituxan®, rituximab, Mabthera®). Rituximab is often used in combination with CHOP stands for Cyclophosphamide, Hydroxydaunorubicin (Adriamycin), Oncovin (Vincristine), Prednisone/Prednisolone.

Thus, in one embodiment, a subject suffering from a B-cell hyperproliferative disorder and having a favorable allele is treated with a therapeutic antibody (e.g. an anti-CD20 antibody, an anti-CD19 antibody, an anti-CD52 antibody, an anti-CD22 antibody) in the absence of a particular adjuvant therapy, optionally wherein the adjuvant therapy comprises chemotherapy (e.g. CHOP in NHL, fludarabine in CLL). In another embodiment, a subject having a favorable allele is treated with an anti-CD20 antibody (e.g. rituximab) as monotherapy, at a standard dose, duration and/or frequency of administration (e.g. using a reference therapeutic regimen), or at a lower dose, duration and/or frequency of administration compared to a reference therapeutic regimen.

In another embodiment, a subject suffering from a B-cell hyperproliferative disorder or other CD20-expressing disorder and having an unfavorable allele is treated with an anti-CD20 antibody (e.g. rituximab) in combination with an adjuvant therapy, optionally wherein the adjuvant therapy comprises chemotherapy (e.g. CHOP in NHL, fludarabine in CLL). In another embodiment, a subject having an unfavorable allele is treated with an anti-CD20 antibody (e.g. rituximab) as monotherapy, at a higher dose and/or higher frequency of administration or for a longer duration compared to a reference therapeutic regimen. In another embodiment, a subject having an unfavorable allele is treated with an antibody (e.g. anti-CD20 antibody) having increased potency, for example with ofatumumab (HuMax-CD20, Genmab A/S), or with an antibody having modified glycosylation such as antibody GA-101 (Roche, Switzerland).

Preparation and dosing schedules for therapeutic agents and chemotherapeutic agents may be used according to manufacturers' instructions or as determined empirically by the skilled practitioner. Preparation and dosing schedules for chemotherapy are also described in, e.g., Chemotherapy Service Ed., M. C. Peny, Williams & Wilkins, Baltimore, Md. (1992) and Lippincott's Cancer Chemotherapy handbook, Baquiran et al, eds. Lippincott, Williams and Wilkins (2002). The chemotherapeutic agent may precede, or follow administration of the antibody or may be given simultaneously therewith. For the prevention or treatment of disease, the appropriate dosage of antibody will depend on the type of disease to be treated, as defined above, the severity and course of the disease, previous therapy, the subject's clinical history and response to the antibody, and the discretion of the attending physician.

Within the context of the present invention, a subject includes any mammalian subject or patient, more preferably a human subject or patient.

Further aspects and advantages of this invention are disclosed in the following experimental section, which should be regarded as illustrative and not limiting the scope of this application.

### Examples

### Materials and Methods

### Patients and Treatment

Clinical trial design, eligibility criteria and end-point assessment have been previously reported (Colombat P, et al. Blood. 2001; 97:101-106). Patients were eligible if they had previously untreated CD20 positive FL with stage II to IV disease and low tumor burden (Brice P, et al. J Clin Oncol. 1997; 15:1110-1117). A total of four 375 mg/m² doses of rituximab (Roche, Neuilly, France) were administered by intravenous infusion (days 1, 8, 15, 22). Clinical response was evaluated at two months (M2) and progression each year until 7 years. Molecular analysis of BCL2-JH gene rearrangement was performed by PCR (Colombat P, et al. 2001), on both peripheral blood (PB) and bone marrow (BM) at diagnosis, M2 and each year. The study protocol was approved by an ethics committee, and all patients gave their informed consent

### Genotyping

Out of the 49 patients included in the clinical trial, two patients refused to be followed and one patient died at 1 year. Forty-six patients were therefore available for genotype analysis. All samples were analysed in the same laboratory and the DNA was extracted using standard procedures. All SNP analyzed generated restriction site (Table 1) and genotyping were therefore performed using a PCR followed by allele-specific restriction enzyme digestion. The primers pairs used for C3, ITGAM-425MT, ITGAM-1130PS and C1QA-70_{GA} were respectively:

(SEQ ID NO: 13)
(C3) 5′-CCAAAACGGCCACCTCGGAA-3′,
(SEQ ID NO: 14)
(C3) 5′-CCGTCCGGCCCACGGGTAGC-3′;
(SEQ ID NO: 15)
(ITGAM-425MT) 5′GAATGCACTTCACCTCTCAGACC-3′,
(SEQ ID NO: 16)
(ITGAM-425MT) 5′-GGGCGCCTCTGTTTGCACATTC-3′;
(SEQ ID NO: 17)
(ITGAM-1130PS) 5′-GCTCTCACTGCCCTCCTCTGC-3′,
(SEQ ID NO: 18)
(ITGAM-1130PS) 5′-GGATACTTCGCTGTCCGAC-3′;
and
(SEQ ID NO: 19)
(C1QA-70_{GA}) 5′-GCCTTAAAGGAGACCAGGGGGAAC-3′,
(SEQ ID NO: 20)
(C1QA-70_{GA}) 5′-CCCTTGAGGAGGAGACGATGGAC-3′.

PCR assays were performed with 10 ng of genomic DNA, 1 μM of each primer, 200 μM of each dNTP (MBI Fermentas, Vilnius, Lithuania) and 1 U of Taq DNA polymerase (Eurobio, Courtaboeuf, France) as recommended by the manufacturer. PCR conditions consisted in 5 min at 94° C. followed by 30 cycles (each consisting in 3 steps at 94° C. for 1 min, 69° C. for 0.5 min, 72° C. for 0.5 min), 40 cycles (each consisting in 3 steps at 94° C. for 1 min, 72° C. for 1 min, 72° C. for 18 sec), 30 cycles (each consisting in 3 steps at 94° C. for 1 min, 71° C. for 0.5 min, 72° C. for 0.5 min) or 30 cycles (each consisting in 3 steps at 94° C. for 1 min, 71° C. for 0.5 min, 72° C. for 0.5 min) for C3-80-RG, ITGAM-425-MT, ITGAM-1130-PS or C1QA-70_{GA} genotyping, respectively. PCR complete extension was achieve for 5 min at 72° C. The amplified DNA (2 mL) was then digested at 37° C. for 2 h with 1 U of HhaI (New England Biolabs, Hitchin, England), NlaIII (New England Biolabs), Avail (Promega, Charbonnière, France) or ApaI (New England Biolabs) for C3-80-RG, ITGAM-425-MT, ITGAM-1130-PS and C1QA-70_{GA} genotyping, respectively. Digested DNA were resolved using standard electrophoresis and visualized under UV light after staining with ethidium bromide. For homozygous C3-80-G, ITGAM-425-T, ITGAM-1130-S and C1QA-70_{G} patients, only one undigested band (430 bp, 198 bp, 200 bp and 281 bp, respectively) was visible. Three bands were seen in heterozygous C3-80-RG (160 bp, 270 bp and 430 bp), ITGAM-425-MT (56 bp, 142 bp and 198 bp), ITGAM-1130-PS (75 bp, 125 bp and 200 bp) and C1QA-70_{GA} (19 bp, 281 bp and 262 bp) patients whereas for homozygous C3-80-R, ITGAM-425-M, ITGAM-1130-P and C1QA-70_{A} patients only two digested bands were obtained (160 bp and 270 bp, 56 bp and 142 bp, 75 bp and 125 bp, 19 bp and 281 bp, respectively).

### Statistical Analysis

Departure of genotype frequencies from Hardy-Weinberg equilibrium was tested by an exact test with the GENOPOP® software (Raymond M, et al. J Heredity. 1995; 86:248-249). Clinical characteristics and clinical responses were compared according to the different genotypes using a Fisher's exact test. A logistic regression analysis including: sex, stage, bone marrow involvement, number of extra-nodal sites, BCL2-JH rearrangement status at diagnosis and genotypes was used to identify independent prognostic variables influencing the clinical response. Progression-free survival (PFS) and overall survival (OS) were calculated using the method of Kaplan and Meier and comparisons by genotype were performed using the log-rank test. A Cox regression including sex, stage, bone marrow involvement, number of extra-nodal sites BCL2-JH rearrangement status at diagnosis and genotypes was performed to identify independent factors influencing PFS and OS. The significance level was P<0.05.

### Results

Out of the 46 patients tested, allele frequencies were: C3-80R: 0.77, C3-80G: 0.33, ITGAM-425-M: 0.84, ITGAM-425-T: 0.16, ITGAM-1130-P: 0.75, ITGAM-1130-S: 0.25, C1QA-70_{A}: 0.67, C1QA-70_{G}: 0.33. Genotype frequencies (Table 2) were similar to those reported elsewhere (Brown K M, et al. N Engl J. Med. 2006; 354:2014-2023; Frenzel H, et al. 2002; Racila D M, et al. 2003) and do not depart from those expected from Hardy-Weinberg equilibrium. There was not significant difference in terms of sex, disease stage, bone marrow involvement, number of extra-nodal sites involved or presence of BCL2-JH rearrangement in peripheral blood and bone marrow at diagnosis according to genotypes (Table 3 and data not shown). The OR rate and survival analyses for the entire cohort with an extended follow-up of 7 years has been already described (Colombat P, et al. 2001; Colombat P, et al. 2006).

According to genotypes, OR rates at M2 was 25% (CR+Cru=25%) and 78% (CR+Cru (complete response, unconfirmed)=28%) for C3-80G homozygous and C3-80R carrier patients, respectively (P=0.042, Table 4). Clinical response was not significantly influenced by other genotypes with OR of 77% and 67% for ITGAM-425M homozygous and heterozygous patients, respectively; 75%, 100% and 72% for ITGAM-1130P homozygous, ITGAM-1130S homozygous and heterozygous patients, respectively; 100%, 72% and 74% for C1QA-70_{G} homozygous, C1QA-70_{A} homozygous and heterozygous patients, respectively. To evaluate the predictive value of polymorphisms we next performed logistic regression. Because we have previously described the influence of FCGR3A-158VF polymorphism on clinical response in this cohort (Cartron G. et al. Blood. 2002; 98:754-758), this parameter was also included in the analysis. The logistic regression analysis showed that C3-80RG polymorphism was the only significant predictive factor for clinical response to rituximab (P=0.042, OR: 0.08, CI 95%: 0.01-0.92).

Median time of PFS (median follow-up: 84 months) was 30 months (CI 95%: 16-51 months) for homozygous ITGAM-425M and 16 months (CI 95%: 6-23 months) for heterozygous patients (P=0.038) whereas PFS was not influenced by C3-80RG, ITGAM-1130PS and C1QA-70_{GA} polymorphisms (data not shown). The Cox regression analysis confirmed the previously described influence of BCL2-JH rearrangement disappearance in BM at D50 (Colombat P, et al. 2001) (P=0.01; HR 0.1, C195%: 0.02-0.65] and showed that ITGAM-425MT polymorphism predicted significantly the PFS (P=0.001; HR: 9.1, CI 95%: 2.4-33.9; FIG. 1). Median time of OS was 72 months (CI 95%: 52-72) for heterozygous ITGAM-425MT patients and was not reached for homozygous ITGAM-425M patients (P=0.07, FIG. 2). BCL2-JH rearrangement status, C3-80RG, ITGAM-1130PS or C1QA-70_{AG} polymorphisms did not influence OS.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

TABLE 1
Single nucleotide polymorphisms (SNP) analyzed in this study

| | | | | | Amino- | |
|---|---|---|---|---|---|---|
| | | | Nucleotide | Amino-acid | acid | Restriction |
| Gene | Location | SNP position | substitution | residue | position | enzyme |
| C3 | 19p13.3-p13.2 | 364 | C | Arg | 80 | Hha I |
| | | (exon 3) | G | Gly | | |
| ITGAM | 16p11.2 | 1420 | T | Met | 425 | Nla III |
| | | (exon 12) | C | Thr | | |
| | | 3534 | C | Pro | 1130 | Ava II |
| | | (exon 30) | T | Ser | | |
| C1QA | 1p36.3-p34.1 | 276 | G | Gly | 70 | Apa I |
| | | (exon 2) | A | Gly | | |

TABLE 2
Genotype frequencies of the population

| Gene | Genotype | Frequency |
|---|---|---|
| C3 | C3-80-RR | 28 (60%) |
| | C3-80-RG | 9 (20%) |
| | C3-80-GG | 9 (20%) |
| ITGAM | ITGAM-425-MM | 31 (67%) |
| | ITGAM-425-MT | 15 (33%) |
| | ITGAM-425-TT | — |
| | ITGAM-1130-PP | 24 (52%) |
| | ITGAM-1130-PS | 21 (46%) |
| | ITGAM-1130-SS | 1 (2%) |
| C1QA | C1QA-70AA | 18 (41%) |
| | C1QA-70AG | 23 (52%) |
| | C1QA-70GG | 3 (7%) |

TABLE 3
Characteristics of patients by C3-80RG and ITGAM-425MT
polymorphisms

| | C3- | C3- | ITGAM- | ITGAM- |
|---|---|---|---|---|
| | 80GG | 80RX | 425MM | 425MT |
| N (%) | 9 (20%) | 37 (80%) | 31 (67%) | 15 (33%) |
| Sex | | | | |
| M | 4 (45%) | 20 (54%) | 14 (45%) | 10 (67%) |
| F | 5 (55%) | 17 (46%) | 17 (55%) | 5 (33%) |
| Disease stage | | | | |
| II-III | 3 (33%) | 11 (30%) | 7 (23%) | 7 (47%) |
| IV | 6 (67%) | 26 (70%) | 24 (77%) | 8 (53%) |
| Bone marrow | | | | |
| involvement | | | | |
| yes | 4 (45%) | 12 (32%) | 8 (26%) | 8 (53%) |
| no | 5 (55%) | 25 (68%) | 23 (74%) | 7 (47%) |
| Extra-nodal | | | | |
| sites involved | | | | |
| <2 | 9 (100%) | 34 (92%) | 29 (93%) | 14 (93%) |
| ≧2 | — | 3 (8%) | 2 (7%) | 1 (7%) |
| BCL2-JH in PB | 5 (55%) | 27 (75%) | 22 (73%) | 10 (67%) |
| BCL2-JH in BM | 4 (50%) | 25 (71%) | 19 (68%) | 10 (67%) |

No patients were homozygous ITGAM-425T

TABLE 4
Clinical response by C3-80R/G polymorphism

| | C3-80GG | C3-80RG | C3-80RR | P |
|---|---|---|---|---|
| Objective | 3 (33%) | 8 (89%) | 23 (82%) | |
| response | | | | |
| complete | 3 (33%) | 2 (22%) | 5 (18%) | |
| remission | | | | |
| complete | 0 | 1 (11%) | 2 (7%) | |
| remission | | | | |
| unconfirmed | | | | |
| partial response | 0 | 5 (56%) | 16 (57%) | 0.004 |
| No response | 6 (67%) | 1 (11%) | 5 (18%) | |
| no change | 5 (56%) | 0 | 5 (18%) | |
| progressive | 1 (11%) | 1 (11%) | 0 | |
| disease | | | | |

1. A cancer prognostic method, the method comprising determining in vitro the genotype of said subject at a polymorphism in the C3-ITGAM axis and making a cancer prognosis of the subject based on said genotype.

2. A method for selection of treatment for a subject having or suspected of having cancer, the methods comprising:
(a) determining the genotype of said subject at a polymorphism in the C3-ITGAM axis,
(b) making a cancer prognosis of the subject based on said genotype; and
(c) subsequent to steps (a)-(b), selecting an anti-cancer treatment for the subject, wherein the selection of treatment is based on the prognosis determined in step (b).

3. The method of claim 2, the method further comprises step (d) treating the subject with the anti-cancer treatment selected in step (c).

4. The method of claim 1, wherein the method comprises determining in vitro the polymorphism in amino acid position 425 for ITGAM.

5. The method of claim 4, comprising determining amino acid residue at position 425 of ITGAM, a methionine (M) at amino acid position 425 being indicative of a favourable cancer prognostic and a threonine (T) at amino acid position 425 being indicative of an unfavourable cancer prognostic.

6. The method of claim 1, the method comprising determining in vitro the polymorphism in amino acid position 80 for C3.

7. The method of claim 6, comprising determining amino acid residue at position 80 of C3, an arginine (R) at amino acid position 80 being indicative of a favourable cancer prognostic and a glycine (G) at amino acid position 80 being indicative of an unfavourable cancer prognostic.

8. The method of claim 1, wherein the prognostic provides a forecast of response to an anti-cancer treatment.

9. The method of claim 8, wherein the anti-cancer treatment comprises administration of a therapeutic antibody.

10. The method of claim 9, wherein the therapeutic antibody comprises an Fc portion of the G1 or G3 subtype.

11. The method of claim 4, wherein determining amino acid residue at position 425 of ITGAM comprises a step selected from the group consisting of:
(a) a step of sequencing the ITGAM gene or RNA or a portion thereof comprising the nucleotides encoding amino acid residue 425;
(b) a step of hybridization of the ITGAM receptor gene or RNA or a portion thereof comprising the nucleotides encoding amino acid residue 425, with a nucleic acid probe specific for the genotype methionine (M) or threonine (T) at amino acid position 425; and
(c) a step of amplifying the ITGAM gene or RNA or a portion thereof comprising the nucleotides encoding amino acid residue 425

12. The method claim 6, wherein determining amino acid residue at position 80 of C3 comprises a step selected from the group consisting of:
(a) a step of sequencing the C3 gene or RNA or a portion thereof comprising the nucleotides encoding amino acid residue 80;
(b) a step of hybridization of the C3 receptor gene or RNA or a portion thereof comprising the nucleotides encoding amino acid residue 80, with a nucleic acid probe specific for the genotype arginine (R) or glycine (G) at amino acid position 80; and
(c) a step of amplifying the C3 gene or RNA or a portion thereof comprising the nucleotides encoding amino acid residue 80.

13. The method of claim 1, wherein the method comprises determining in vitro the polymorphism in amino acid position 425 for ITGAM or determining in vitro the polymorphism in amino acid position 80 for C3, and wherein determining amino acid residue at position 425 of ITGAM or position 80 of C3 comprises a step of sequencing the ITGAM or C3 protein or a portion thereof comprising the amino acid at residue 425 of ITGAM or residue 80 of C3, respectively.

14. The method of claim 6, wherein determining amino acid residue at position 80 of C3 comprises (a) contacting a sample from a subject with an affinity reagent specific for an ITGAM polypeptide having an M or T at amino acid position 425, or a C3 polypeptide having an R or G at amino acid position 80; and (b) detecting the ITGAM or C3 affinity reagent -ITGAM or -C3 polypeptide complex in the sample.

15. The method of claim 11, wherein amplification is performed by polymerase chain reaction (PCR), such as PCR, RT-PCR and nested PCR.

16. The method of claim 13, wherein determining amino acid residue at position 425 of ITGAM or the amino acid at position 80 of C3 comprises a step of allele-specific restriction enzyme digestion.

17. The method of claim 4, wherein determining amino acid residue at position 425 of ITGAM comprises:
obtaining genomic DNA from a biological sample,
amplifying the ITGAM gene or a portion thereof comprising the nucleotides encoding amino acid residue 425, and
determining the amino acid residue at position 425 of said ITGAM.

18. The method of claim 4, wherein determining amino acid residue at position 425 of ITGAM comprises:
obtaining genomic DNA from a biological sample,
amplifying the ITGAM gene or a portion thereof comprising the nucleotides encoding amino acid residue 425,
introducing an allele-specific restriction site,
digesting the nucleic acids with the enzyme specific for said restriction site and,
analysing the digestion products, e.g., by electrophoresis, the presence of digestion products being indicative of the presence of the allele.

19. The method of claim 6, wherein determining amino acid residue at position 80 of C3 comprises:
obtaining genomic DNA from a biological sample,
amplifying the C3 gene or a portion thereof comprising the nucleotides encoding amino acid residue 80, and
determining the amino acid residue at position 80 of said C3 gene.

20. The method of claim 6, wherein determining amino acid residue at position 80 of C3 comprises:
obtaining genomic DNA from a biological sample,
amplifying the C3 gene or a portion thereof comprising the nucleotides encoding amino acid residue 80,
introducing an allele-specific restriction site,
digesting the nucleic acids with the enzyme specific for said restriction site and,
analysing the digestion products, e.g., by electrophoresis, the presence of digestion products being indicative of the presence of the allele.

21. The method of claim 1, wherein the subject has a B-cell lymphoproliferative disorder.

22. The method of claim 21, wherein the disorder is a NHL.

23. The method of claim 3, wherein the anti-cancer treatment comprises treatment with a therapeutic antibody.

24. The method of claim 23, wherein said therapeutic antibody is an anti-CD20 antibody.

25. The method of claim 24, wherein said anti-CD20 antibody is rituximab.

26. A method for treatment for a subject having or suspected of having cancer, the method comprising:
(a) determining the genotype of said subject at a polymorphism at amino acid position 425 in ITGAM,
(b) predicting a response of the subject to treatment with a therapeutic antibody based on said genotype, wherein an M at amino acid position 425 is indicative of a favourable cancer prognostic and a T at amino acid position 425 is indicative of an unfavourable cancer prognostic;
(c) subsequent to steps (a)-(b), selecting an anti-cancer treatment for the subject, wherein the selection of treatment is based on the prognosis determined in step (b); and
(d) treating the subject with the anti-cancer treatment selected in step (c).

27. A method for treatment for a subject having or suspected of having cancer, the method comprising:
(a) determining the genotype of said subject at a polymorphism at amino acid position 80 in C3,
(b) predicting a response of the subject to treatment with a therapeutic antibody based on said genotype, wherein an arginine (R) at amino acid position 80 being indicative of a favourable cancer prognostic and a glycine (G) at amino acid position 80 being indicative of an unfavourable cancer prognostic;
(c) subsequent to steps (a)-(b), selecting an anti-cancer treatment for the subject, wherein the selection of treatment is based on the prognosis determined in step (b); and
(d) treating the subject with the anti-cancer treatment selected in step (c).

28. The method of claim 26, wherein step (d) comprises treating the subject determined to have an unfavourable cancer prognostic with a therapeutic antibody in combination with an adjuvant.

29. The method of claim 26, wherein step (d) comprises treating the subject determined to have an unfavourable cancer prognostic with a therapeutic antibody having increased potency, optionally wherein the therapeutic antibody comprises a hypofucosylated Fc portion.

30. The method of claim 26, wherein step (d) comprises treating the subject determined to have a favourable cancer prognostic with a therapeutic antibody in the absence of an adjuvant.

31. Use of a therapeutic antibody for the treatment of a subject having or suspected of having cancer, the subject having a genotype at a polymorphism in ITGAM or C3 indicative of a favourable cancer prognostic, wherein the therapeutic antibody is administered in a therapeutic regimen specially adapted to a subject having a genotype at a polymorphism in ITGAM or C3 indicative of a favourable cancer prognostic.

32. The use of claim 31, wherein therapeutic regimen comprises treatment with a therapeutic antibody in the absence of an adjuvant.

33. Use of a therapeutic antibody for the treatment of a subject having or suspected of having cancer, the subject having a genotype at a polymorphism in ITGAM or C3 indicative of an unfavourable cancer prognostic, wherein the therapeutic antibody is administered in a therapeutic regimen specially adapted to a subject having a genotype at a polymorphism in ITGAM or C3 indicative of an unfavourable cancer prognostic.

34. The use of claim 33, wherein therapeutic regimen comprises treatment with a therapeutic antibody in the presence of an adjuvant.

35. The use of claim 33, wherein therapeutic regimen comprises treatment with a therapeutic antibody having increased potency, optionally wherein the therapeutic antibody comprises a hypofucosylated Fc portion.
